SEQUENCE LISTING (5 sequences)

SEQ ID NO: 1 - DNA, 525 bp, Ornithodoros moubata
  CDS (13)..(519)
  sig_peptide (13)..(66)
taagagctcaaaatgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctaggcggcc

SEQ ID NO: 2 - protein, 168 aa, Ornithodoros moubata
  misc_feature: Protein sequence including signal peptide at residues 1 to 18
MLVLVTLIFSFSANIAYADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 3 - protein, 150 aa, Ornithodoros moubata
  misc_feature: Protein sequence lacking N terminal signal peptide
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 4 - DNA, 525 bp, Artificial sequence
  SEQ ID NO 1 altered by site directed mutagenesis.
taagagctcaaaatgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagcaaggcacagactgggcttcaaccgattggacgtttactttggacggcgcaaaggtaacggcaacccttggtcaactaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctaggcggcc

SEQ ID NO: 5 - protein, 168 aa, Artificial sequence
  SEQ ID NO 4 altered by site directed mutagenesis.
MLVLVTLIFSFSANIAYADSESDCTGSEPVDAFQAFSEGKEAYVLVRSTDPKARDCLKGEPAGEKQDNTLPVMMTFKQGTDWASTDWTFTLDGAKVTATLGQLTQNREVVYDSQSHHCHVDKVEKEVPDYEMWMLDAGGLEVEVECCRQKLEELASGRNQMYPHLKDC

(19) United States
(12) Patent Application Publication
(10) Pub. No.: US 2011/0059885 A1
(43) Pub. Date: Mar. 10, 2011

(54) TREATMENT OF DISEASES AND CONDITIONS MEDIATED BY EICOSANOIDS

(75) Inventors: Susan Lea, Oxford (GB); Miles Nunn, Oxford (GB); Pietro Roversi, Oxford (GB)

(73) Assignee: Natural Environment Research Council

(21) Appl. No.: 12/866,024

(22) PCT Filed: Feb. 5, 2009
(86) PCT No.: PCT/GB09/00311

§ 371 Date: Nov. 8, 2010

(30) Foreign Application Priority Data
Feb. 5, 2008 (GB) 0802116.4

(51) Int. Cl. A61K 38/17; C07K 14/435; C07H 21/00; A61K 31/7088; A61P 17/00; A61P 35/00; A61P 11/00; A61P 35/04; A61P 11/06; A61P 9/10; A61P 9/00; A61P 19/02; A61P 13/12; A61P 29/00; A61P 17/06; A61P 37/02; A61P 25/28; A61P 25/00

(52) U.S. Cl. 514/1.4; 530/350; 536/23.5; 514/1.1; 514/44 R; 514/21.2; 514/18.6; 514/19.3; 514/1.8; 514/19.8; 514/1.7; 514/1.9; 514/16.4; 514/16.8; 514/15.4; 514/16.6

(57) ABSTRACT

The method of the invention relates to an OmCI polypeptide or a polynucleotide encoding an OmCI polypeptide for the treatment of a disease or condition mediated by a leukotriene or hydroxyeicosanoid.

### FIELD OF THE INVENTION

The present invention relates to compositions useful in the treatment of diseases and conditions mediated by eicosanoids and in particular to tick-derived inhibitors of complement for treatment of diseases and conditions mediated by leukotrienes and hydroxyeicosanoids.

### BACKGROUND OF THE INVENTION

Eicosanoids are a family of oxygenated biologically active lipid mediators derived from the 20-carbon fatty acid arachidonate (AA) through three major enzymatic pathways: cyclooxygenase (COX), lipoxygenase (LO), and cytochrome P450 monooxygenase (CYP450). Eicosanoids include prostanoids (including prostaglandins, PGs, and thromboxanes, TXBs) derived from COX pathway, leukotrienes from LO pathway and hydroxyeicosatetraenoic acids (HETEs) and epoxyeicosatrienoic acids (EETs) from LO and P450 monooxygenase pathways (Curtis-Prior, 2004; Peters-Golden & Henderson Jr., 2007). Eicosanoids mediate numerous effects on diverse cell types and organs. These effects include regulation of vascular tone and permeability of capillaries and venules (PGs, TBXs, LKs), contraction or relaxation of muscle (PGs, TBXs, cysteinyl LKs), stimulation or inhibition of platelet function (TBXs, PGs), regulation of renal blood flow and mineral metabolism (Imig, 2000; Hao and Breyer, 2007), control of growth and or spread of malignant cells (Schwartz et al., 2005; Aya, 2006), and activation of leukocytes in particular in autoimmune and inflammatory conditions (LKs, HETEs) (Samuelsson, 1983; Kim & Luster, 2007).

LTB4 and the hydroxyeicosanoids mediate their effects though the BLT1 and BLT2 G-protein coupled receptors (Yokomizo et al., 1997, 2000). Human BLT1 is a high affinity receptor (Kd 0.39-1.5 nM; Tager and Luster, 2003) specific for LTB₄ with only 20-hydroxy LTB4 and 12-epi LTB4 able to displace LTB4 in competitive binding studies (Yokomizo et al., 2001). Human BLT2 has a 20-fold lower affinity (Kd 23 nM) for LTB4 than BLT1 and is activated by binding a broader range of eicosanoids including 12-epi LTB4, 20-hydroxy LTB4, 12(S)- and 15(S)-HETE and 12(S)- and 15(S)-HPETE (Yokomizo et al., 2001). Human BLT2 has 45.2 and 44.6% amino acid identity with human and mouse BLT1, while human and mouse BLT2 have 92.7% identity (Yokomizo et al., 2000).

Human BLT1 is mainly expressed on the surface of leukocytes, though it has recently been described in endothelial cells and vascular smooth muscle cells. Human BLT2 is expressed in a broader range of tissue and cell types. A number of specific antagonists of BLT1 and BLT2 have been described which inhibit activation, extravasation and apoptosis of human neutrophils (Kim and Luster, 2007) and reduce symptoms caused by neutrophil infiltration in mouse models of inflammatory arthritis (Kim et al., 2006) and renal ischaemia reperfusion (Noiri et al., 2000). Increasing numbers of studies indicate that both BLT1 and BLT2 can mediate pathological effects through LTB4 and hydroxyeicosanoids (Lundeen et al., 2006), although BLT1 certainly has a dominant role in some pathologies such as collagen induced arthritis in mice (Shao et al., 2006). BLT1−/− deficient mice have also highlighted the importance of BLT1 in directing neutrophil migration in inflammatory responses. In particular, a 5LO deficient mouse strain was used to show autocrine activation of BLT1 on neutrophils is needed for their recruitment into arthritic joints (Chen et al., 2006).

LTB₄ is the most powerful chemotactic and chemokinetic eicosanoid described and promotes adhesion of neutrophils to the vascular endothelium via upregulation of integrins (Hoover et al., 1984). It is also a complete secretagogue for neutrophils, induces their aggregation and increases microvascular permeability. LTB₄ recruits and activates natural killer cells, monocytes and eosinophils. It increases superoxide radical formation (Harrison et al., 1995) and modulates gene expression including production of a number of proinflammatory cytokines and mediators which may augment and prolong tissue inflammation (Ford-Hutchinson, 1990; Showell et al., 1995). LTB4 is increasingly being shown to have roles in the induction and management of adaptive immune responses. For example regulation of dendritic cell trafficking to draining lymph nodes (Klaas et al., 2006; Del Prete et al., 2007), Th2 cytokine IL-13 production from lung T cells (Miyahara et al., 2006), recruitment of antigen-specific effector CD8+ T cells (Taube et al., 2006) and activation and proliferation of human B lymphocytes (Yamaoka et al., 1989).

Oxidised isomeric derivatives of LTB₄ such as B4 isoleukotrienes are also biologically active (Harrison et al., 1995). As are the hydroxyeicosanoids, for example 5(S)-HETE is a highly potent chemoattractant for eosinophils (Powell and Rokach, 2005). The cysteinyl LKs, which are derived from LTA₄, are correlated with the pathophysiology of asthma, including: bronchoconstriction caused by contraction of smooth muscle lining the airways; mucosal edema caused by vascular leakage; increased secretion of mucus; and the presence of an inflammatory-cell infiltrate that is rich in eosinophils (Bisgaard et al., 1985; Drazen et al., 1988).

A number of marketed drugs target the eicosanoids. These include the glucocorticoids which modulate phopholipase A2 (PLA₂) and thereby inhibit release of the eicosanoid precursor AA (Sebaldt et al., 1990). Non-steroidal anti-inflammatory drugs (NSAID) and other COX2 inhibitors which prevent synthesis of the prostaglandins and thromboxanes (Curry et al., 2005). There are also a number of LK modifiers which either inhibit the 5-LO enzyme required for LTB4 synthesis (Zileuton; Dube et al., 1998), or antagonise the CysLT₁ receptor that mediates the effects of cysteinyl leukotrienes (Zafirlukast and Montelukast) (Sharma and Mohammed, 2006). The LK modifiers are orally available and have been approved by the FDA for use in the treatment of chronic asthma. Montelukast has also received FDA approval for seasonal allergic rhinitis in January 2003 and exercise-induced bronchoconstriction (EIB) in April 2007. An intravenous formulation of Montelukast, which will have a rapid onset of clinical effect compared to the oral formulation, is being developed for the treatment of acute asthma. Montelukast is not approved for use in cystic fibrosis though there is some evidence of therapeutic effect (Stellmach et al., 2005).

WO2004/106369 describes soft tick derived complement (C) inhibitor OmCI that inhibits both the classical and alternative complement pathways by direct binding to complement component C5 (Nunn et al., 2005). OmCI is derived from the salivary glands of haemotophagous anthropods. It has proven therapeutic potential (Hepburn et al., 2007).

### SUMMARY OF THE INVENTION

It has now been shown that OmCI binds to eicosanoids. In particular, the invention relates to the previously unproven ability of OmCI to bind eicosanoids in particular LKs, especially leukotriene B₄ (LTB4) and the hydroxyeicosanoid 12(S)-hydroxyeicosatetraenoic acid (HETE). OmCI in unmodified or modified form may also bind 12-epi LTB4, 20-hydroxy LTB4, and other hydroxyeicosanoids including 15(S)-hydroxyeicosatetraenoic acid (HETE) and 12(S)- and 15(S)-hydroperoxyeicosatetrenoic acid (HPETE). The invention also relates to the use of OmCI in the treatment and prevention of diseases where leukotrienes, especially LTB₄ and hydroxyeicosanoids are implicated in pathology. OmCI binds to and cages LKs and hydroxyeicosanoids. This may prevent the ligands interacting with both the BLT1 and BLT2 receptors and ameliorate the proinflammatory effects of the fatty acids which have frequently been shown to depend on signalling through both receptors.

Thus in accordance with one aspect of the present invention, there is provided an OmCI polypeptide or a polynucleotide encoding an OmCI polypeptide for the treatment of a disease or condition mediated by a leukotriene or hydroxyeicosanoid.

In accordance with a preferred embodiment of the present invention, the OmCI polypeptide comprises:
(a) an amino acid sequence of SEQ ID NO: 3;
(b) a variant thereof having at least 60% identity to the amino acid sequence of SEQ ID NO: 3 and leukotriene (LK/E) binding activity; or
(c) a fragment of either thereof having LKJE binding activity.

The polypeptides or polynucleotides of the present invention may be used in the treatment of inflammatory diseases or conditions and other diseases and conditions mediated by a leukotriene or hydroxyeicosanoid. Examples of diseases and disorders which can be treated in accordance with the present invention include uveitis, atopic dermatitis, contact hypersensitivity, ulcerative colitis, oesophygeal adenocarcinoma, pancreatic adenocarcinoma, breast cancer, ovarian cancer, acne, aneurysm, periodontal disease, cystic fibrosis, prostate cancer, asthma, atherosclerosis, psoriasis, bronchiolitis and inflammatory bowel disease.

In another aspect of the present invention, there is provided a method of treating or preventing a disease or condition mediated by a leukotriene or hydroxyeicosanoid in a subject in need thereof, the method comprising administering to a subject a therapeutically effective amount of an OmCI polypeptide or a polynucleotide encoding an OmCI polypeptide.

In further aspect of the present invention, there is provided a composition comprising an OmCI polypeptide and a fatty acid. The fatty acid is preferably a therapeutic fatty acid and is provided for delivery to an individual.

### DESCRIPTION OF THE FIGURES

FIG. 1: Detail from crystal structure of bacterial expressed OmCI (bOmCI) bound to palmitoleic acid (centre of picture).

FIG. 2: Enzyme immunoassay (EIA) showing binding of 12(S)-HETE by 41.2 μg bOmCI, pg/mL 12(S)-HETE in solution following 20 min preincubation of 12500 pg/mL 12(S)-HETE with or without PBS, OmCI, RaHBP2.

FIG. 3: Dose dependence of 12(S)-HETE binding by bOmCI, 12(S)-HETE in solution following incubation with 12500 pg/mL 12(S)-HETE and 3 concentrations of bOmCI.

FIG. 4: Effect of preincubation time of bOmCI with 12(S)-HETE, longer preincubation time has no effect on 12(S)-HETE binding by bOmCI.

FIG. 5: Binding of 12(S)-HETE by 41.2 mcg of bOmCI, yOmCI and RaHBP2, EIA showing bOmCI captures more 12(S)-HETE than an equivalent amount of yeast expressed yOmCI.

FIG. 6: TXB2 in solution following incubation of OmCI and RaHBP2 with 3333 pg/mL TXB2, EIA showing absence of binding to thromboxane B₂ by bOmCI.

FIG. 7: Apparent LTB4 in solution following incubation of bOmCI and RaHBP2 with 750 pg/mL LTB4, EIA showing apparent concentration of LTB₄ in presence of bOmCI.

FIG. 8: Dose dependence of LTB₄-AP binding by bOmCI, dilution series bOmCI with LTB4-AP conjugate.

FIG. 9: Comparison of LTB4-AP binding by equivalent concentrations of bOmCI and yOmCI (8.6 mg/mL stocks), dose dependent binding of LTB₄ by yOmCI and bOmCI is similar.

FIG. 10: Effect of excess 12(S)-HETE binding of LTB4-AP binding to 8.24 μg bOmCI, excess 12(S)-HETE does not out compete LTB₄ binding to bOmCI.

FIG. 11: LTB4 (spheres) docked in the OMCI model PDB ID 2CM4 (sticks) after removing water molecule Z23 that was filling in the bottom of the pocket (Z23 was H-bonded to the main chain carbonyls of E41 and F36).

FIG. 12: OmCI ablates lesions formed in response to 100 ng LTB₄ applied to skin. The photograph was taken 23 hours post application. Scale bar shown.

FIG. 13: Absorption spectra of bOMCI and LTB₄. (A) LTB₄ in solution (upper line) before addition of OmCI, and re-measurement of same solution (lower line) after addition then removal of the bOmCI:LTB4 complex by ultrafiltation (B) bOmCI:LTB₄ complex (upper line) and bOmCI (lower line) only after concentration to 200 μl by ultrafiltation.

FIG. 14: Detail from the crystal structure of bOmCI bound to LTB₄ (centre of picture). Oxygen atoms in LTB₄, at carboxy-group and hydroxyl-groups at C-5 and C-12, are shown. These groups form hydrogen bonds (dotted lines) with amino acids in the binding cavity (see text example 7).

FIG. 15: At 4:1 to 1:1 molar ratios OmCI but not OmCI pre-loaded with LTB₄ ablates lesions formed in response to 100 ng LTB₄ applied to skin. The photograph was taken 48 hours post application. Scale bar shown.

FIG. 16: Intravenous administration of 50 μg OmCI (referred to as EV576 in FIG. 16) reduces neutrophil recruitment in the lung and decreases vascular permeability and protein exudation resulting from the intranasal administration of 150 g anti-ovalbumin (Ova) antibody.

### DESCRIPTION OF THE SEQUENCES

SEQ ID NO: 1 is the polynucleotide and encoded protein sequence of OmCI of Ornithodoros moubata.

SEQ ID NO: 2 is the amino acid sequence of OmCI Ornithodoros moubata.

SEQ ID NO: 3 is the amino acid sequence of amino acids 19 to 168 shown in SEQ ID NO: 2 and is the amino acid sequence of OmCI without the first amino acid sequences of the protein of SEQ ID NO: 2, which is a signal sequence.

SEQ ID NO: 4 and 5 are the polynucleotide and encoded protein sequence and protein sequence respectively of OmCI, modified to change Asn78 to Gln and Asn 102 to Gln, with a codon change from AAT and AAC respectively to CAA, for expression in yeast to avoid hyperglycosylation.

Thus, the present invention provides an OmCI polypeptide or a polynucleotide encoding an OmCI polypeptide for the treatment of a disease or condition mediated by leukotrienes or hydroxyeicosanoids. The OmCI protein may be a tick-derived complement inhibitor, isolated from the saliva of Ornithodoros moubata or may be a functional equivalent thereof, including homologues thereof and fragments of either thereof.

The OmCI protein of the present invention is preferably OmCI from Ornithodoros moubata. This protein was first isolated from the salivary glands of the tick and has been found to inhibit the classical and alternative complement pathways. The amino acid sequence for this protein is shown in SEQ ID NO: 2. A polypeptide according to the invention may include the complete sequence shown in SEQ ID NO: 2. In the alternative, the polypeptide is provided which does not include the first 18 amino acids of the protein sequence which form a signal sequence. Accordingly, a polypeptide according to the invention can be that of SEQ ID NO: 3, that is amino acids 19 to 168 of the amino acid sequence of SEQ ID NO: 2.

A variant, such as a homologue, or fragment of the OmCI protein from Ornithodoros moubata is also provided in accordance with the invention. Such homologues may include paralogues and orthologues of the OmCI sequence that is set out in SEQ ID NO: 2 or 3, including, for example, the OmCI protein sequence from other tick species including Rhipicephalus appendiculatus, R. sanguineus, R. bursa, A. americanum, A. cajennense, A. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, A. reflexus, Ornithodoros erraticus, O. moubata moubata, O. m. porcinus, and O. savignyi. The term “homologue” is also meant to include the OmCI protein sequence from mosquito species, including those of the Culex, Anopheles and Aedes genera, particularly Culex quinquefasciatus, Aedes aegypti and Anopheles gambiae; flea species, such as Ctenocephalides fells (the cat flea); horseflies; sandflies; blackflies; tsetse flies; lice; mites; leeches; and flatworms.

In one embodiment, the OmCI polypeptide comprises:
(a) the amino acids sequence of SEQ ID NO: 3;
(b) a variant thereof having at least 60% identity to the amino acid sequence of SEQ ID NO: 3 and having LK/E binding activity; or
(c) a fragment of either thereof having LK/E binding activity.

Variant polypeptides are those for which the amino acid sequence varies from that in SEQ ID NO: 2 or 3, but which retain the same essential character or basic functionality of LK/E binding as OmCI.

LK/E binding activity as used herein refers to the ability to bind to leukotrienes and hydroxyeicosanoids including but not limited to LTB4, B4 isoleukotrienes and any hydroxylated derivative thereof, HETEs, HPETEs and EETs.

The variant polypeptides may therefore display LK/E binding activity. Typically, polypeptides with more than about 50%, 55% or 65% identity, preferably at least 70%, at least 80%, at least 90% and particularly preferably at least 95%, at least 97% or at least 99% identity, with the amino acid sequence of SEQ ID NO: 2 or 3 are considered variants of the protein. Such variants may include allelic variants and the deletion, modification or addition of single amino acids or groups of amino acids within the protein sequence, as long as the peptide maintains the basic functionality of OmCI. The identity of variants of SEQ ID NO: 3 may be measured over a region of at least 50, at least 100, at least 130 or at least 140 or more contiguous amino acids of the sequence shown in SEQ ID NO: 3, or more preferably over the full length of SEQ ID NO: 3.

Amino acids that are particularly likely to be required for LK/E binding include (with reference to SEQ ID NO. 2): Phe36, Arg 54, Leu57, Gly59, Val72, Met74, Phe76, Thr85, Trp87, Phe89, Gln105, Arg107, His119, Asp121, Trp133

Amino acid identity may be calculated using any suitable algorithm. For example the UWGCG Package provides the BESTFIT program which can be used to calculate homology (for example used on its default settings) (Devereux et al (1984) Nucleic Acids Research 12, 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The variant sequences typically differ by at least 1, 2, 3, 5, 10, 20, 30, 50 or more mutations (which may be substitutions, deletions or insertions of amino acids). For example, from 1 to 50, 2 to 40, 3 to 30 or 5 to 20 amino acid substitutions, deletions or insertions may be made. The substitutions are preferably conservative substitutions, for example according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The fragment of the OmCI polypeptide used in the invention is typically at least 50, for example at least 80 or more amino acids in length, up to 90, 100, 120, 130 or 140 amino acids in length, as long as it retains the LK/E binding activity of OmCI.

The polypeptides of the invention may also be provided as a fusion protein comprising an OmCI polypeptide genetically or chemically fused to another peptide. The purpose of the other peptide may be to aid detection, expression, separation or purification of the protein. Alternatively the protein may be fused to a peptide such as an Fc peptide to increase the circulating half life of the protein. Examples of other fusion partners include beta-galactosidase, glutathione-S-transferase, or luciferase.

The polypeptides used in the invention may be chemically modified, e.g. post-translationally modified. For example, they may be glycosylated, pegylated, phosphorylated or comprise modified amino acid residues. They may be modified by the addition of histidine residues to assist their purification or by the addition of a signal sequence to promote insertion into the cell membrane. Such modified polypeptides fall within the scope of the term “polypeptide” used herein.

Typically, a polypeptide for use in accordance with the invention displays LK/E binding activity. Indeed OmCI has a propensity to bind to any non-cyclic fatty acid of between 16 and 20 carbon atoms in length. Certain fatty acids, in particular LTB4 bind more tightly than others. Other fatty acids to which the polypeptide of the invention may bind include arachidonic acid, 12-epi LTB4, 20-hydroxy LTB4 and the hydroxyeicosanoids including 12(S)-hydroxyeicosatetraenoic acid (HETE) and 12(S)-hydroperoxyeicosatetraenoic acid (HPETE). The LK/E binding activity or binding activity to other fatty acids of the polypeptide may be determined by means of a suitable assay such as enzyme immunoassays, mass spectrometry or radioligand or fluorescently labelled ligand binding assays that are familiar to those skilled in the art. One such binding assay is exemplified in the Examples. In some embodiments, it may be preferable to select a polypeptide that preferentially binds to a specific fatty acid, such as LTB4. Such preferential binding activity can be determined by suitable assays, for example, competition assays as exemplified in the Examples.

In accordance with some aspects of the invention, preferably, a polypeptide for use in accordance with the invention retains the complement inhibitor activity shown by OmCI of Ornithodoros moubata. Preferably, the polypeptide inhibits both the classical and the alternative pathways of complement activation. By inhibit is meant that the effect of the alternative and classical pathways of complement activation is reduced. The ability of a molecule to reduce the effect of the classical complement pathway and the alternative complement pathway can be determined by standard haemolytic assays known in the art such as those described in Giclas et al. (1994) and in WO2004/106369. Preferably, the presence of a complement inhibitor polypeptide of the invention reduces red blood cell lysis in standard haemolytic assays for the classical and alternative pathways of complement activation by at least 20% compared to a standard assay in the absence of a complement inhibitor polypeptide, more preferably by at least 30%, 40%, 50%, 60%, 70% or 80%.

Preferably, the complement inhibitor polypeptide inhibits cleavage of C5 by the C5 convertase in the classical pathway and the C5 convertase in the alternative pathway. The conversion of C5 to C5b by C5 convertase occurs in both the alternative complement pathway and the classical complement pathway. The C5 convertase in the classical pathway is C4b3b2a and the C5 convertase in the alternative pathway is C3b2Bb. The inhibition of C5 cleavage by both these C5 convertases thus inhibits both the classical and alternative pathways of complement activation. The ability of a molecule to inhibit cleavage of C5 by the C5 convertases of the classical and alternative pathways can be determined by standard in vitro assays. Preferably, the presence of a complement inhibitor polypeptide reduces cleavage of C5 by the C5 convertases of the classical and alternative pathways by at. least 20% compared to a standard assay in the absence of a complement inhibitor polypeptide, more preferably by at least 30%, 40%, 50%, 60%, 70% or 80%. Preferably, the complement inhibitor activity of the polypeptides of the inventions inhibits cleavage of C5 by the C5 convertases of the classical and alternative pathways from a range of mammalian species.

In another aspect of the invention, the OmCI polypeptides for use in accordance with the invention are selected such that the complement inhibitor activity is reduced or absent. For example, the OmCI polypeptide can be mutated in the 132 to 142 loop (by reference to SEQ ID No 1) which is the beta H to C terminal alpha2 helix. For example the one or more or all of the amino acids in the loop can be deleted or substituted with amino acids for example from TSGP2 to reduce or remove binding for C5, and thus having reduced complement inhibitor activity.

Polypeptides for use in the invention may be in a substantially isolated form. It will be understood that the polypeptide may be mixed with carriers or diluents which will not interfere with the intended purpose of the polypeptide and still be regarded as substantially isolated. A polypeptide for use in the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention.

Polypeptides for use in the present invention may be isolated from any tick that produces an OmCI polypeptide or a variant of an OmCI polypeptide.

Polypeptides for use in the invention may also be prepared as fragments of such isolated polypeptides. Further, the OmCI polypeptides may also be made synthetically or by recombinant means. For example, a recombinant OmCI polypeptide may be produced by transfecting mammalian, fungal, bacterial or insect cells in culture with an expression vector comprising a nucleotide sequence encoding the polypeptide operablylinked to suitable control sequences, culturing the cells, extracting and purifying the OmCI polypeptide produced by the cells.

The amino acid sequence of polypeptides for use in the invention may be modified to include non-naturally occurring amino acids or to increase the stability of the compound. When the polypeptides are produced by synthetic means, such amino acids may be introduced during production. The polypeptides may also be modified following either synthetic or recombinant production.

Polypeptides for use in the invention may also be produced using D-amino acids. In such cases the amino acids will be linked in reverse sequence in the C to N orientation. This is conventional in the art for producing such polypeptides.

A number of side chain modifications are known in the art and may be made to the side chains of the OmCI polypeptides, provided that the polypeptides retain LK/E binding activity.

### Polynucleotides

A polynucleotide encoding an OmCI polypeptide or variant may be used to treat or prevent a disease or condition mediated by leukotrienes or eicosanoids. In particular the polynucleotide may comprise or consist of: (a) the coding sequence of SEQ ID NO: 1; (b) a sequence which is degenerate as a result of the genetic code to the sequence as defined in (a); (c) a sequence having at least 60% identity to a sequence as defined in (a) or (b) and which encodes a polypeptide having LK/E binding activity; or (d) a fragment of any one of the sequences as defined in (a), (b) or (c) which encodes a polypeptide having LK/E binding activity.

Typically the polynucleotide is DNA. However, the polynucleotide may be a RNA polynucleotide. The polynucleotide may be single or double stranded, and may include within it synthetic or modified nucleotides.

A polynucleotide of the invention can typically hybridize to the coding sequence or the complement of the coding sequence of SEQ ID NO: 1 at a level significantly above background. Background hybridization may occur, for example, because of other DNAs present in a DNA library. The signal level generated by the interaction between a polynucleotide of the invention and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, Molecular Cloning: A Laboratory Manual, 1989). For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 1 may be modified by nucleotide substitutions, for example from 1, 2 or 3 to 10, 25, 50 or 100 substitutions. The polynucleotide of SEQ ID NO: 1 may alternatively or additionally be modified by one or more insertions and/or deletions and/or by an extension at either or both ends. Additional sequences such as signal sequences may also be included or sequences encoding another peptide or protein to aid detection, expression, separation or purification of the protein or encoding a peptide such as an Fc peptide to increase the circulating half life of the protein. Examples of other fusion partners include beta-galactosidase, glutathione-S-transferase, or luciferase.

The modified polynucleotide generally encodes a polypeptide which has LK/E binding activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 3 over a region of at least 20, preferably at least 30, for instance at least 40, at least 60, at least 100, at least 200, at least 420, or most preferably over the full length of SEQ ID NO: 1 or the length of SEQ ID NO: 1 encoding a polypeptide having the sequence shown in SEQ ID NO: 1. Sequence identity may be determined by any suitable method, for example as described above.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 60, preferably over 100 nucleotides forms one aspect of the invention, as does a polynucleotide which has at least 95% sequence identity over 420 nucleotides.

Polynucleotide fragments will preferably be at least 20, for example at least 25, at least 30 or at least 50 nucleotides in length. They will typically be up to 100, 150, 250 or 400 nucleotides in length. Fragments can be longer than 400 nucleotides in length, for example up to a few nucleotides, such as five, ten or fifteen nucleotides, short of the coding sequence of SEQ ID NO: 1.

Polynucleotides for use in the invention may be produced recombinantly, synthetically, or by any means available to those of skill in the art. They may also be cloned by standard techniques. The polynucleotides are typically provided in isolated and/or purified form.

In general, short polynucleotides will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15-30 nucleotides) to a region of the OmCI gene which it is desired to clone, bringing the primers into contact with DNA obtained from an arthropod cell performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the OmCI gene sequence described herein. Although in general the techniques mentioned herein are well known in the art, reference may be made in particular to Sambrook et al. (1989).

OmCI polynucleotides as described herein have utility in production of the polypeptides for use in the present invention, which may take place in vitro, in vivo or ex vivo. The polynucleotides may be used as therapeutic agents in their own right or may be involved in recombinant protein synthesis.

The polynucleotides for use in the invention are typically incorporated into a recombinant replicable vector. The vector may be used to replicate the nucleic acid in a compatible host cell. Therefore, polynucleotides for use in the invention may be made by introducing an OmCI polynucleotide into a replicable vector, introducing the vector into a compatible host cell and growing the host cell under conditions which bring about replication of the vector. The host cell may, for example, be an E. coli cell.

Preferably the vector is an expression vector comprising a nucleic acid sequence that encodes an OmCI polypeptide. Such expression vectors are routinely constructed in the art of molecular biology and may for example involve the use of plasmid DNA and appropriate initiators, promoters, enhancers and other elements, such as for example polyadenylation signals, which may be necessary and which are positioned in the correct orientation in order to allow for protein expression. The coding sequences may also be selected to provide a preferred codon usage suitable for the host organism to be used. Other suitable vectors would be apparent to persons skilled in the art. By way of further example in this regard we refer to Sambrook et al. (1989).

Preferably, a polynucleotide for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vector is typically adapted to be used in vivo.

Promoters and other expression regulation signals may be selected to be compatible with the host cell for which expression is designed. Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences. This will allow the introduction of the polynucleotides of the invention into the genome of eukaryotic cells by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

### Diseases and Conditions

The present inventors have found that OmCI has the ability to bind to LTB4 and the hydroxyeicosanoid 12(S)-HETE. LTB4 is the most powerful chemotatic and chemokinetic eicosanoid described and promotes adhesion of neutrophils to the vascular endothelium via up-regulation of integrins. LTB4 induces aggregation of neutrophils and through a variety of processes plays a role inflammation. LTB4 has been shown to have roles in the induction and management of adaptive immune responses. Thus, OmCI, having the ability to bind to and cage leukotrienes and hydroxyeicosanoids can prevent these ligands interacting with BLT1 and BLT2 receptors and can be used to ameliorate the proinflammatory effects of the fatty acids.

Examples of specific disorders that can be treated in accordance with the present invention include uveitis, atopic dermatitis, contact hypersensitivity, ulcerative colitis, oesophygeal adenocarcinoma, pancreatic adenocarcinoma, breast cancer, ovarian cancer, colon cancer, lung cancer, acne, obliterative bronchiolitis, aneurysms, periodontal disease, cystic fibrosis, prostate cancer, post-inflammatory pigmentation, fibromyalgia, systemic lupus erythematosus, tumor metastasis, sclerodermia, multiple sclerosis, sarcoidosis, radiation induced gastrointestinal inflammation, and gout.

Further conditions and disorders that can be treated in accordance with the present invention include asthma, bronchitis, atherosclerosis, psoriasis, psoriatic arthritis, inflammatory bowel disease (including Crohn's disease), sepsis, arteritis, myocardial infarction, stroke, and coronary heart disease, ischaemia reperfusion injury, nephritis and arthritis, including rheumatoid arthritis, spondyloarthropathies, osteoarthritis, and juvenile arthritis.

Conditions known to be mediated by LTB4 that can be treated in accordance with the present invention include obliterative bronchiolitis, scleroderma interstitial lung disease, periodontal disease, chronic B lymphocytic leukaemia, prostate cancer and atherosclerosis.

Conditions known to be mediated by LTB4 and complement that can be treated in accordance with the present invention include nephritis, arthritis of various sorts, uveitis, cancer, sepsis, ischaemia reperfusion injury, stroke and myocardial infarction.

Conditions in which anti-inflammatory fatty acids (such as lipoxins and resolvins) are known to play a role and which might be delivered by OmCI in accordance with the present invention include scleroderma interstitial lung disease, fibrosis, periodontal disease, arthritis, asthma, atherosclerosis and colitis.

### Therapy and Prophylaxis

The present invention provides the use of OmCI polypeptides and polynucleotides to treat or prevent a disease or condition mediated by leukotrienes and eicosanoids. Treatment may be therapeutic or prophylactic.

The OmCI polypeptide or polynucleotide may be administered to an individual in order to prevent the onset of one or more symptoms of the disease or condition. In this embodiment, the subject may be asymptomatic. The subject may have a genetic predisposition to the disease. A prophylactically effective amount of the polypeptide or polynucleotide is administered to such an individual. A prophylactically effective amount is an amount which prevents the onset of one or more symptoms of a disease or condition.

A therapeutically effective amount of the OmCI polypeptide or polynucleotide is an amount effective to ameliorate one or more symptoms of a disease or condition. Preferably, the individual to be treated is human.

The OmCI polypeptide or polynucleotide may be administered to the subject by any suitable means. The polypeptide or polynucleotide may be administered by enteral or parenteral routes such as via oral, buccal, anal, pulmonary, intravenous, intra-arterial, intramuscular, intraperitoneal, intraarticular, topical or other appropriate administration routes.

The OmCI polypeptide or polynucleotide may be administered to the subject in such a way as to target therapy to a particular site.

The formulation of any of the polypeptides and polynucleotides mentioned herein will depend upon factors such as the nature of the polypeptide or polynucleotide and the condition to be treated. The polypeptide or polynucleotide may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally, topically or by infusion techniques. The polypeptide or polynucleotide may also be administered as suppositories. A physician will be able to determine the required route of administration for each particular patient.

Typically the polypeptide or polynucleotide is formulated for use with a pharmaceutically acceptable carrier or diluent and this may be carried out using routine methods in the pharmaceutical art. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Pharmaceutical compositions suitable for delivery by needleless injection, for example, transdermally, may also be used. The compositions according to the invention may be presented in all dosage forms normally used for topical application, in particular in the form of aqueous, aqueous-alcoholic or, oily solutions, of dispersions of the lotion or serum type, of anhydrous or lipophilic gels, of emulsions of liquid or semi-solid consistency of the milk type, obtained by dispersing a fatty phase in an aqueous phase (O/VV) or vice versa (VV/O), or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

They may also be used for the scalp in the form of aqueous, alcoholic or aqueous-alcoholic solutions, or in the form of creams, gels, emulsions or foams or alternatively in the form of aerosol compositions also containing a propellant agent under pressure.

The amounts of the different constituents of the compositions according to the invention are those traditionally used in the fields in question.

A therapeutically effective amount of polypeptide or polynucleotide is administered. The dose may be determined according to various parameters, especially according to the polypeptide or polynucleotide used; the age, weight and condition of the patient to be treated; the route of administration; and the required regimen. Again, a physician will be able to determine the required route of administration and dosage for any particular patient. A typical daily dose is from about 0.001 to 50 mg per kg, preferably from about 0.01 mg/kg to 10 mg/kg of body weight, according to the activity of the polypeptide, the age, weight and conditions of the subject to be treated, the type and severity of the disease and the frequency and route of administration. Preferably, daily dosage levels are from 0.5 mg to 2 g. Lower dosages may be used for topical administration.

The OmCI nucleotide sequences described above and expression vectors containing such sequences can also be used as pharmaceutical formulations as outlined above. Preferably, the nucleic acid, such as RNA or DNA, in particular DNA, is provided in the form of an expression vector, which may be expressed in the cells of the individual to be treated. The formulations may comprise naked nucleotide sequences or be in combination with cationic lipids, polymers or targeting systems. The formulations may be delivered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

OmCI polypeptides have been shown to bind to any non-cyclic fatty acids of between 16 and 20 carbon atoms in length. However, LTB4 is bound more tightly than other fatty acids. The OmCI polypeptides of the present invention can also be used to deliver other fatty acids, for example, fatty acids which have therapeutic activity. OmCI polypeptides of the invention can be used to target such fatty acids to the site of inflammation, in the presence of LTB4. In particular, OmCI polypeptides will release bound fatty acid in the presence of LTB4. Thus, OmCI can be used to target a desired fatty acid to a site of inflammation.

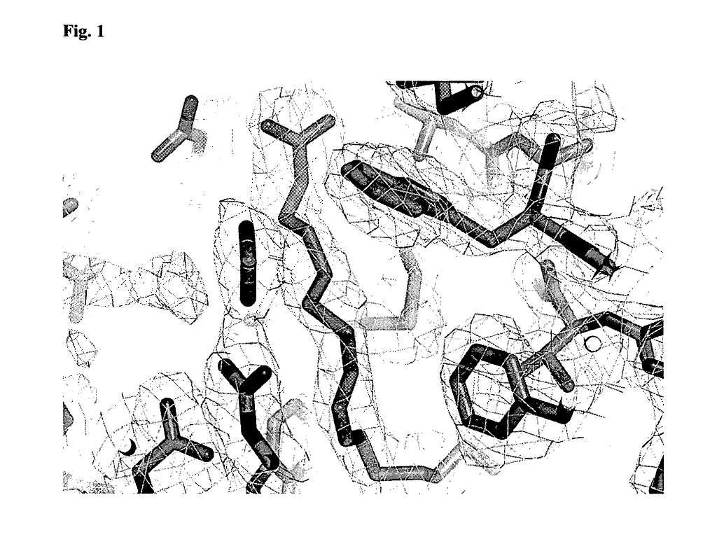

For example, OmCI expressed in yeast was found to contain ricinoleic acid in its binding pocket (Roversi et al., 2007) and OmCI expressed in bacteria bound palmitoleic acid (FIG. 1). Anti-inflammatory fatty acids can therefore be loaded into a OmCI polypeptide of the invention. On contact with the site of inflammation, containing LTB4, the bound fatty acid can be released, by displacement by LTB4 which binds to the OmCI polypeptide more tightly. Examples of therapeutic fatty acids that can be used in accordance with this aspect of the invention include lipoxin A4, lipoxin B4, resolvins, protectins,15(S)-HETE, docosatrienes, 13-hydroxyoctadecadienoic acid, 15-hydroxyeicosatrienoic acid,15-hydroxyeicosapentaenoic acid, 17-hydroxydocosahexaenoic acid, ricinoleic acid, and nitrated fatty acids and analogues of all thereof (Bannenberg et al., 2005; Cui et al., 2006; McMahon and Godson, 2004; Papayianni et al., 1996; Serhan et al., 2000 and 2002; Serhan and Savill, 2005; Ternowitz et al., 1989; Takata et al., 1994). Lipoxins, for example, are endogenously produced anti-inflammatory agents that modulate leukocyte trafficking and stimulate nonphlogistic macrophage phagocytosis of apoptotic neutrophils which promote the resolution of inflammation. In a preferred embodiment, the fatty acids selected for targeting by OmCI do not possess a hydroxyl group on C15 of the carbon chain. Alternatively, a modified OmCI can be used, for example in which Arg 107 of SEQ ID NO. 2 is modified, for example to Gly, to avoid steric interference in the binding pocket when binding to fatty acids having a hydroxyl group on C15, or when binding to lipoxins.

### Examples

### Example 1

### OmCI Binds 12(S)-HETE (12(S)-hydroxyeicosatetraenoic Acid) in a Competitive ELISA

### Background:

OmCI binds to fatty acids (FIG. 1). Mass spectroscopy shows that ricinoleic acid (C₁₈H₃₄O₃) and palmitoleic acid (C₁₆H₃₀O₂) are the predominant forms found in OmCI expressed in P. methanolica and E. coli respectively. However, the true physiological ligands are more likely to be one or more of the many host cell membrane derived eicosanoids which mediate inflammation, oxidative stress and cell signalling.

Competitive enzyme immunoassays (EIAs) from Assay Designs Inc. are available for the quantification of a number of the eicosanoids. One such EIA kit uses a polyclonal antibody to 12(S)-HETE to bind 12(S)-HETE labelled with alkaline phosphatase and competing unlabelled 12(S)-HETE in the sample or standards of known concentration. After simultaneous incubation at room temperature and capture of the antibody on the plate, the excess reagents are washed away, the substrate added and reaction measured by microplate reader. The higher the concentration of 12(S)-HETE in sample or standard the lower the absorbance reading, because the unlabelled fatty acid competes for binding with the alkaline phosphatase labelled molecule.

We hypothesised that OmCI would compete for binding with the eicosanoid specific antibodies used in the immunoassays. 12(S)-hydroxyeicosatetraenoic acid (12(S)-HETE) was chosen to test this idea since it is (perhaps) the eicosanoid with the most similar physicochemical characteristics to ricinoleic acid (which, from our crystallographic data, is predicted to bind more tightly than palmitoleic acid). Among other effects, 12(S)-HETE has been shown to be chemotactic and chemokinetic for polymorphonuclear leukocytes and vascular smooth muscle cells.

### Methods:

12(S)-HETE EIA Kit was from Assay Designs (Cat. No. 900-050). OmCI stocks used were expressed either in yeast (yOmCI) or bacteria (bOmCI). Both stocks were ≧98% pure, 8.3 mg/mL in phosphate buffered saline pH 7.2 (PBS). The negative control tick histamine binding protein RaHBP2, which is also a lipocalin (Paesen et al., 1999), was expressed in bacteria and was also ≧98% pure, 8.3 mg/mL in PBS. 12(S)-HETE standard was diluted to 50000, 12500, 3125, 781, 195 pg/mL in the assay buffer supplied with the kit. 100 μl of the 12500, 3125 and 0 pg/mL solutions were mixed with ≦9 μl of phosphate buffered saline pH 7.2 (PBS) or solutions of OmCI or RaHBP2 in PBS. The mixtures were incubated at room temperature for 20 minutes then used in the 12(S)-HETE immunoassay in accordance with the manufacturers instructions. The absorbance readings of the treated samples were compared with a standard curve to estimate the concentration of 12(S)-HETE available in solution for binding by the anti-12(S)-HETE polyclonal antibody.

### Results:

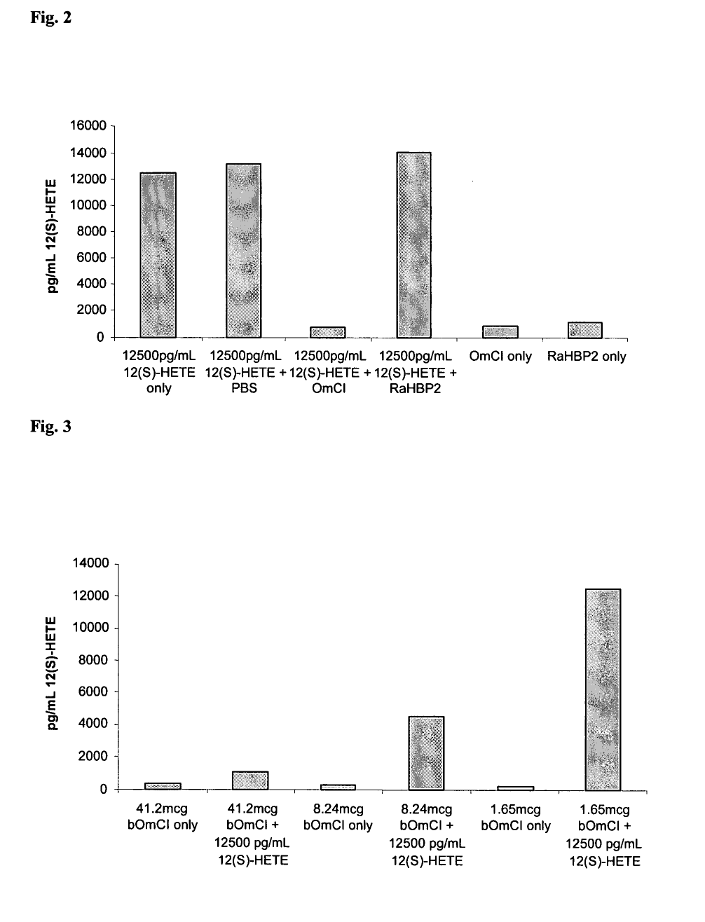

bOmCI but not RaHBP2 decreases the amount of 12(S)-HETE available in solution for antibody binding suggesting bOmCI binds directly to 12(S)-HETE (FIG. 2). PBS and both the bOmCI and RaHBP2 purified protein preps appear to contain some (≦1000 pg/mL) 12(S)-HETE.

### Discussion:

These initial results with the bacterially expressed protein suggest OmCI can bind fatty acids that are longer (C20) and have a greater number of unsaturated bonds (four) than either palmitoleic (C16 and 1 double bond) or ricinoleic acid (C18 and 1 double bond). Furthermore 12(S)-HETE does not have a double bond at C9-C10 which was predicted to be important for ligand binding. The results also suggest that palmitoleic acid can be displaced from the binding pocket of bOmCI by 12(S)-HETE. Although caution is needed with this assumption, since OmCI was used in large molar excess (˜1000-4000-fold) and it is possible that a proportion of the purified bOmCI is not occupied by any ligand.

### Example 2

### Parameters that Affect 12(S)-HETE Binding to OmCI

### Methods:

Similar methods to those described in Example 1 were used.

### Results and Discussion

A large molar excess of bOmCI to 12(S)-HETE is needed to give results that unambiguously demonstrate 12(S)-HETE binding (FIG. 3). In the assay shown in FIG. 3, bOmCI is in approximately 634, 127 and 25.5 molar excess to 12(S)-HETE. The need for a significant molar excess of OmCI may reflect competition between bOmCI and anti-12(S)-HETE antibody for 12(S)-HETE binding, low affinity binding of 12(S)-HETE by bOmCI, and/or binding only by bOmCI that is not occupied by palmitoleic acid.

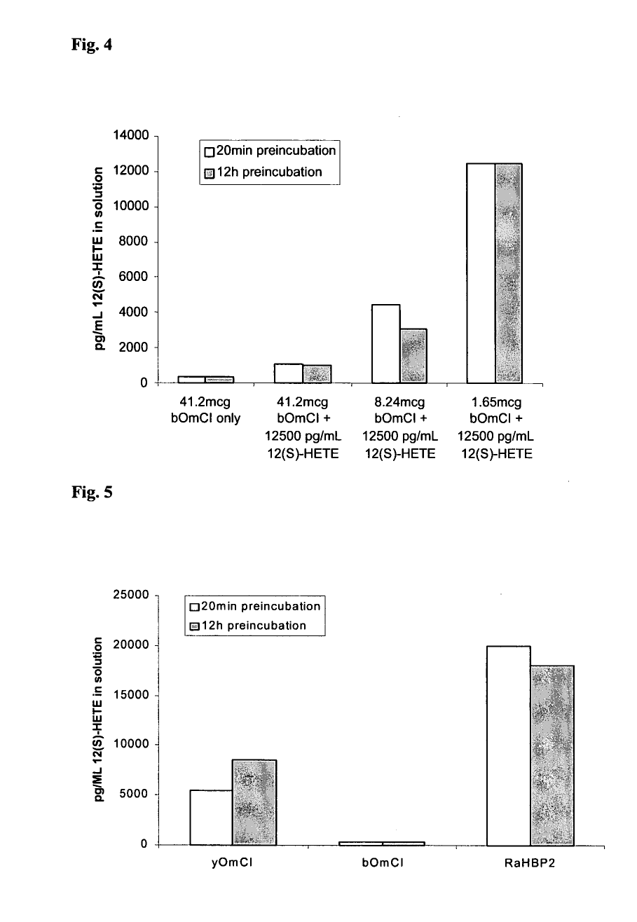

Prolonged incubation (overnight at room temperature) does not increase the proportion of 12(S)-HETE bound by bOmCI (FIG. 4).

At equivalent concentrations, yeast (y) OmCI binds less 12(S)-HETE than bOmCI (FIG. 5). At 634 molar excess OmCI to 12(S)-HETE yOmCI binds roughly 50% of and bOmCI 97% of 12500 pg/mL 12(S)-HETE. For unknown reasons in this experiment, the control (RaHBP2) gives an estimated concentration of about 19000 pg/mL 12(S)-HETE rather than the expected 12500 pg/mL.

These observations suggest that 12(S)-HETE is captured by recombinant bOmCI that is empty and may displace a proportion of the palmitoleic acid from bOmCI and yOmCI. Ricinoleic acid, which occupies all the binding pockets in yOmCI crystals, appears to be displaced to a more limited extent by 12(S)-HETE. This is consistent with our crystallography based prediction that ricinoleic acid binds more tightly to OmCI than palmitoleic acid.

### Example 3

### OmCI Binds to LTB₄, but not TXB₂ or the Cysteinyl Leukotrienes

### Method:

Assay Design Inc. EIA kits for solution measurement of leukotriene B₄ (LTB₄), thromboxane B₂ (TXB₂) and the cysteinyl leukotrienes (cys-LKs) were purchased and used in accordance with the manufacturer's instructions. 100 μl of the standard solutions were mixed with ≦90 of PBS or diluted stock solutions of OmCI or RaHBP2. The mixtures were incubated at room temperature for 20 minutes then used in the immunoassays in accordance with the manufacturers instructions. The absorbance readings of the treated samples were compared with a standard curve to estimate the concentration of eicosanoids available in solution for binding by the anti-eicosanoid polyclonal antibodies.

### Results:

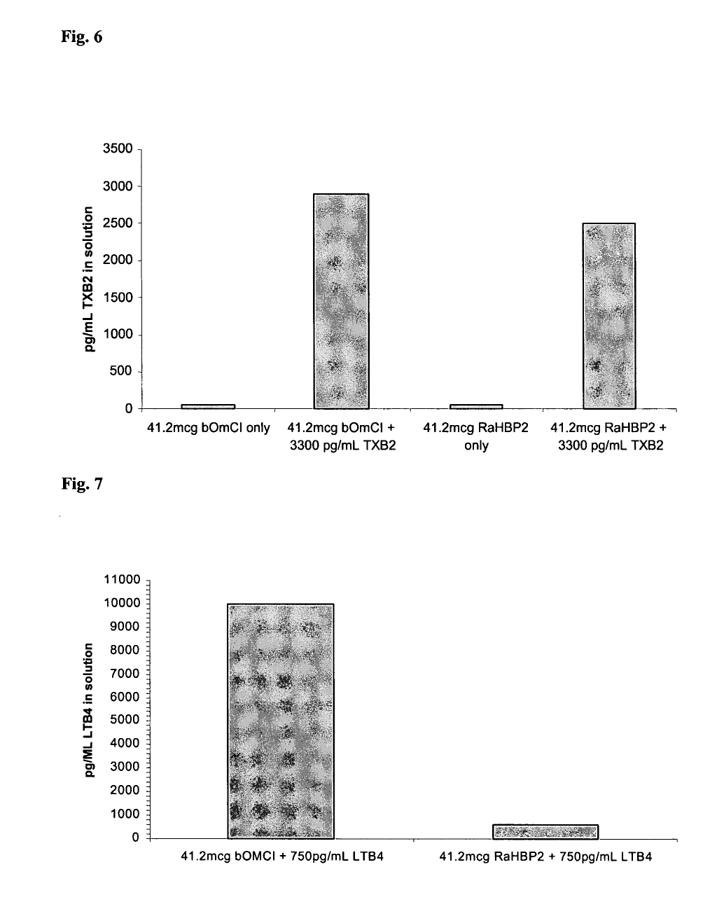

bOmCI does not appear to bind the cyclic eicosanoid TXB2 (FIG. 6) or the amino acid conjugated Cys-LKs (data not shown). This agrees with our crystallographic data which shows the binding pocket of OmCI is not large enough to accommodate these ligands (Roversi et al., 2007).

The first experiment using the LTB₄ EIA kit (FIG. 7) suggested that bOmCI binds directly to the LTB4-alkaline phosphatase (AP) conjugate since OD readings were effectively zero and thus the estimated concentration of LTB₄ in solution (10000 pg/mL) was much higher than the amount of LTB4 actually added to the assay (750 pg/mL). The amount of LTB4 detected in the assay using the control protein RaHBP2 was 610 pg/mL LTB4; which is similar to the actual amount of LTB4 that was added to the assay.

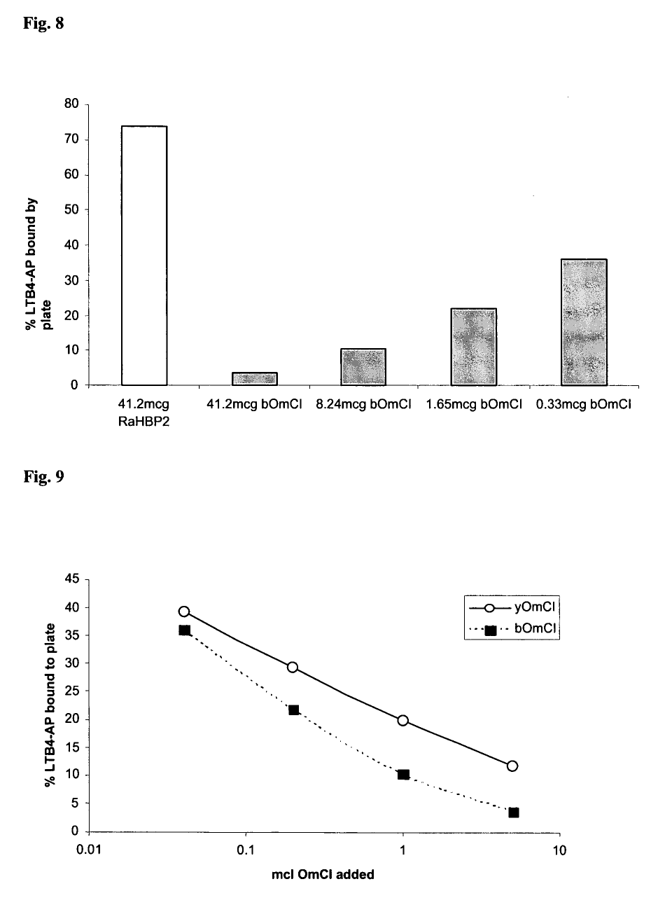

To examine the possibility that bOmCI binds directly to the LTB4-AP conjugate, the assay was performed using only 50 μL LTB4-AP conjugate as ligand and omitting unlabelled LTB4. FIG. 8 shows dose dependent binding of bOmCI to LTB4-AP. 12(S)-HETE-, TXB2- and cys-LK-AP conjugates were not bound directly by bOmCI (data not shown).

The kit manufacturers do not know the concentration of LTB₄-AP in the kits they sell. However, if we assume, an unrealistically high, 100% conjugation efficiency and assume IC50=1:1 binding then from standard curves the concentration of LTB₄-AP may be approximately 110 μg/mL. From FIG. 8 we can see that 0.33 μg bOmCI binds approximately 50% of the LTB₄-AP. From this we can calculate that a 1200× excess of bOmCI is needed to bind 50% of the LTB₄-AP. This may seem a large excess but binding is undertaken in the presence of anti-LTB4 antibody and binding to the conjugate may be impaired by the linker.

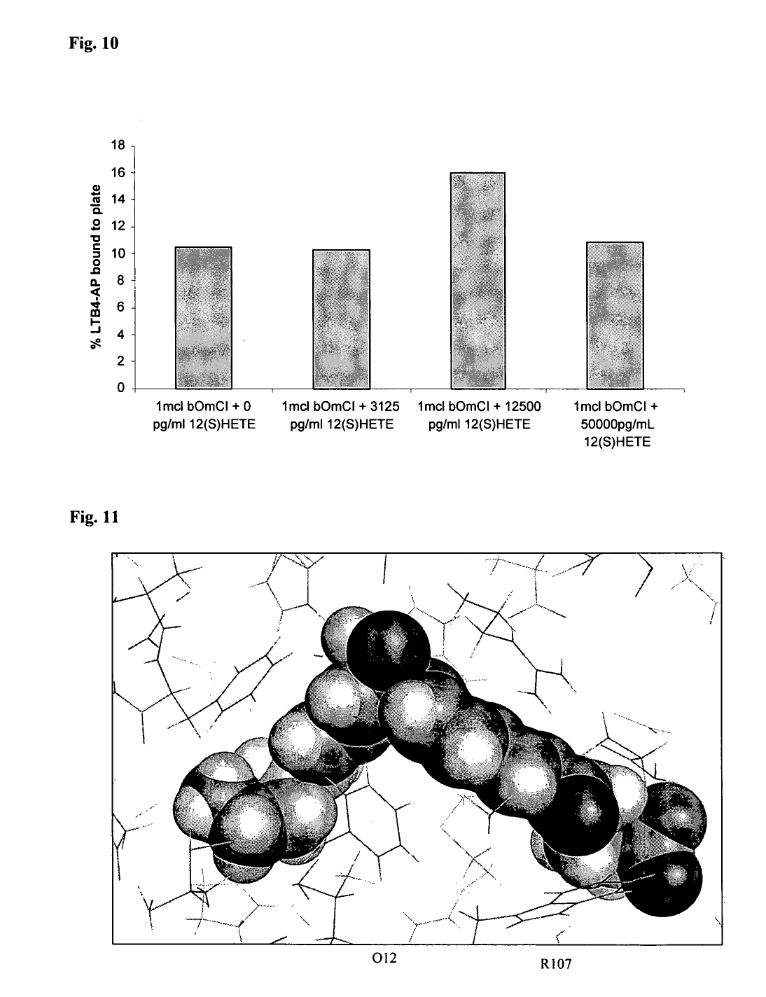

Binding of LTB₄-AP to yOmCI and bOmCI is fairly similar (FIG. 9) which suggests that, unlike 12(S)-HETE (FIG. 5), LTB4-AP is able to displace ricinoleic acid from the binding pocket moderately efficiently. Excess 12(S)-HETE does not outcompete LTB₄-AP binding to bOmCI (FIG. 10). In this experiment, if excess 12(S)-HETE displaced LTB₄-AP from bOmCI the % of LTB₄-AP bound to LTB₄ specific antibody on captured on the plate would increase, and it does not.

### Example 4

### Theoretical Modelling Shows that LTB4 Fits Neatly in the Binding Pocket of OmCI

### Method:

An atomic model for LTB4 was constructed by using the PRODRG server at: http://davapc1.bioch.dundee.ac.uk/programs/prodrg/. The first 18 atoms of this LTB4 model were then manually fitted to the ricinoleic acid molecule in PDB ID 2CM4, and the 2 extra C atoms of the LTB4 tail rotated so as to point into the bottom of the OMCI pocket—after removal of the water molecule Z23 that fills that space in the crystal. This OMCI:LTB4 model was then idealised/optimized with geometric constraints only, using the programs BUSTER-TNT and CCP4-REFMAC5.

### Results:

The C20 chain of LTB4 in the fatty acid binding pocket of OmCI can be accommodated (FIG. 11) by the removal of water Z23 from the PDB deposited structure (PDB ID 2CM4). The water obviously was filling in the pocket when ricinoleic acid, which has a shorter C18 chain, was bound. The water molecule forms hydrogen bonds to the carbonyl groups of amino acids E41 and F36. Exchange of a longer fatty acid for a shorter fatty acid in the binding pocket would be favoured by entropy by the removal of the water molecule.

### Example 5

### The Local Skin Reaction Induced by Topical Application of LTB4 is Ablated by the Addition of Recombinant OmCI

### Background

Topical application of more than 5 ng LTB4 to human skin induces local erythema and oedema (Greaves, 1984). Reactions appear after 12 hours and peak at 24-48 hours.

### Method:

2 μL LTB4 (50 ng/μL stock in pure ethanol from Biomol International, LP) were mixed with 28 μL PBS containing approximately 33×, 16×, 7×, or 2× molar excess of bOmCI (17 kDa) or 15×, 7×, 3×, 1× negative control protein ovalbumin (Mr 45 kDa) and incubated for 20 minutes at room temperature. The solutions were then applied to the flexor surface of the forearm and air dried. The most concentrated protein solutions used (including 2 μL pure ethanol) and LTB4 were also applied on their own. The deposits were occluded under a chamber which was removed after 6 hours. Skin reactions were observed from 12-96 hours.

### Results:

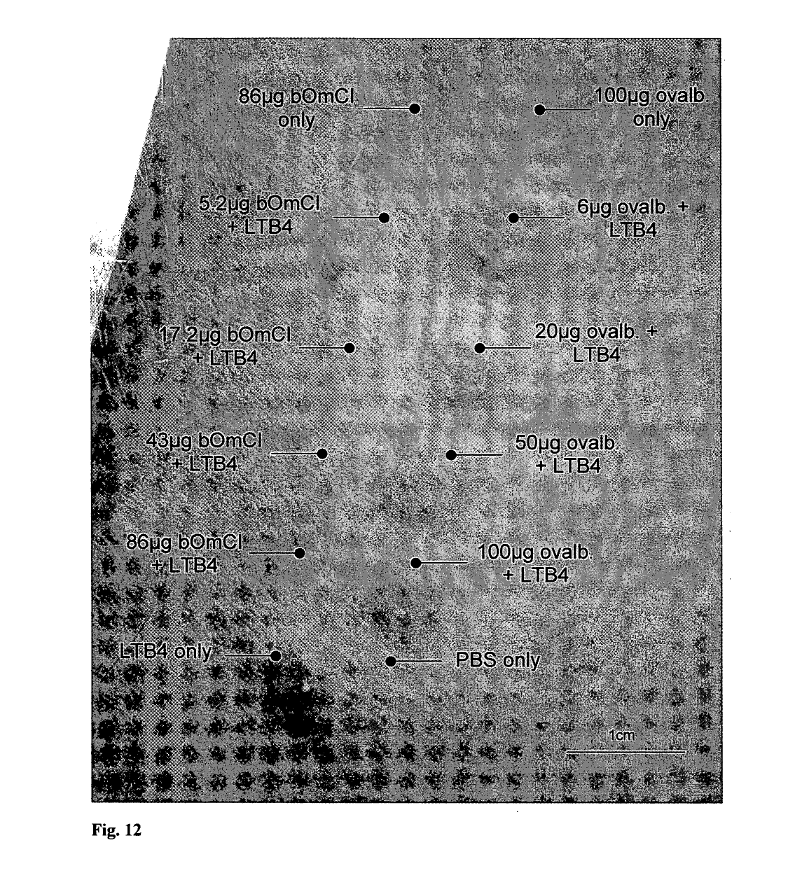

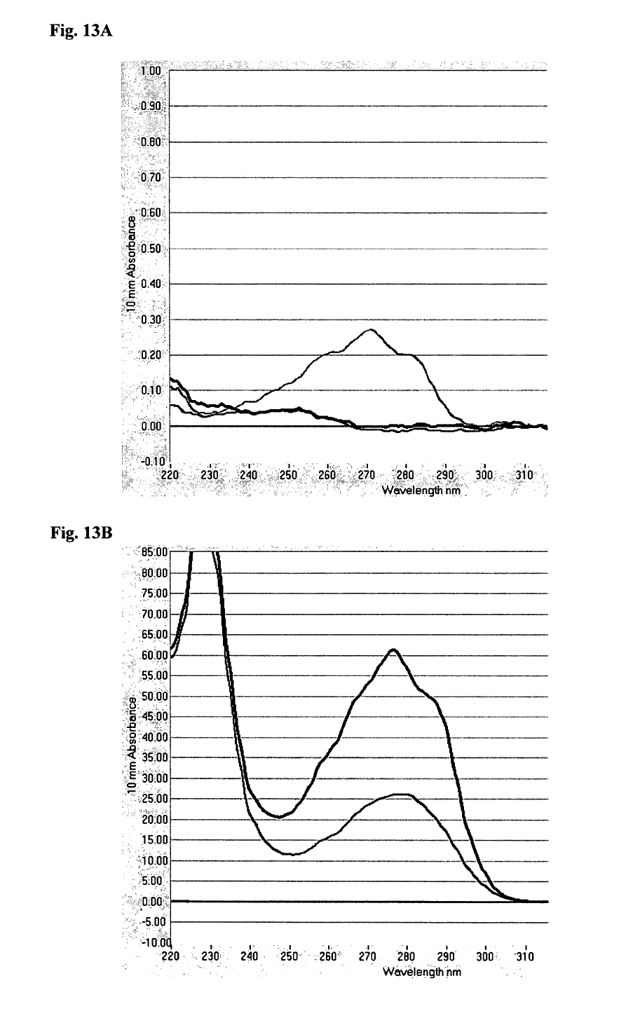

As shown in FIG. 12 OmCI ablated the skin reaction induced by topical application of 100 ng LTB4. Reactions were maximal at 20-30 hours post application. The skin reaction was completely ablated by all the four concentrations of OmCI that were tested. Ovalbumin had no effect on lesion formation compared to LTB4 alone. The proteins applied without LTB4 did not induce skin reactions. The results indicate that bOmCI binds LTB4 in solution and prevents its absorption through intact skin.

### Example 6

### LTB4 Binding by OmCI is Evident by Absorbance

### Background:

Leukotrienes have characteristic, strong, UV absorption spectra due to their conjugated double bond systems (the triene chromophore). In aqueous media LTB₄ has a peak absorbance at 271 nm and ‘shoulders at 262 nm and 282.5 nm. Protein peak absorbance is at 280 nm. OmCI bound to LTB₄ should exhibit increased UV absorbance at around 280 nm, compared to the protein on its own, and LTB₄'s characteristic shoulders l Onm either side of the peak absorbance.

### Method:

bOmCI (4.5 mg) was incubated with 1.8 mL LTB4 (50 ng/μL stock in pure ethanol, Biomol International) in 39 mL PBS at room temperature with shaking for 10 minutes. This mixture is a 1:1 molar ratio between OmCI and LTB4. The mixture was concentrated to 200 μl in Vivaspin (Sartorious) 5 kDa cut off ultrafiltration device. The retentate was washed with a further 30 mL of PBS and concentrated to 200 μl. In parallel, the same amount (4.5 mg) of bOmCI was incubated with 1.8 ml ultrapure ethanol in 39 mL PBS, then concentrated and washed as described above. The final volume of the concentrated proteins was 200 μl. UV absorption spectra of the proteins were examined using a Nanodrop ND-1000 Spectrophotometer.

### Results:

The spectra obtained are shown in FIG. 13. LTB4 alone has the characteristic absorbance peaks expected in phosphate buffered saline pH 7.4 with peaks at 271, 261 and 281 nm (FIG. 13A). The absorption spectra of bOmCI incubated with LTB₄, washed extensively to remove residual LTB₄, has shoulders indicative of LTB₄ binding and peak absorbance is significantly higher than bOmCI incubated with pure ethanol (FIG. 13B). This indicates that bOmCI selectively binds LTB₄ and removes it from solution. Indeed, no LTB₄ is detectable in the flow through from the initial ultrafiltation step (FIG. 13A) which indicates that (within the limits of detection) all the LTB4 added to initial mixture was bound by the bOmCI.

Significant changes in the UV spectra of LTB₄ bound by bOmCI were observed. The UV maximum exhibited a +6 nm bathochromic (red shift) to 277, 267 and 287 nm (FIGS. 13A and B). The shift is most likely caused by dispersion interactions between the conjugated leukotriene and bOmCI amino acids. This is consistent with the triene chromophore being completely encompassed by the protein. Similar interactions will cause hypochromism of UV absorption by the triene chromophore. This was not measured directly, but it is notable that peak absorption expected from input LTB₄ concentrated to 200 μl (final volume of concentrated protein) would be 55.8 (calculation 41.32 ml/0.2 ml×0.27 10 mm Absorbance) whereas total peak absorption of LTB₄ bound to bOmCI was approximately 35.07 (calculation peak 10 mm absorption of bOmCI:LTB4 minus peak absorption bOmCI i.e. 61.19-26.12). Assuming minimal losses of protein, the calculation implies hypochromism.

### Example 7

### Crystallographic Structural Data Shows LTB4 in the Binding Pocket of bOmCI

### Method:

bOmCI protein loaded with LTB₄ was made as described above (Example 6), then concentrated to 25 mg/mL, buffer exchanged to Tris-HCl pH 7, 30 mM NaCL and used to grow crystals. A diffraction dataset was collected from a P2₁ OmCI:LTB4 monoclinic crystal (a=41.76 Å b=112.81 Å c=62.40 Åβ=101.89°, 4 copies/asymmetric unit) in July 2008 on BM14@ESRF. The data have been processed to 2.0 Å resolution, the structure was initially determined by molecular replacement and the OmCI:LTB4 model built and refined to R=20.7 R_{free}=23.7, rmsd_{bonds}=0.005, rmsd_{angles}=0.9.

### Results:

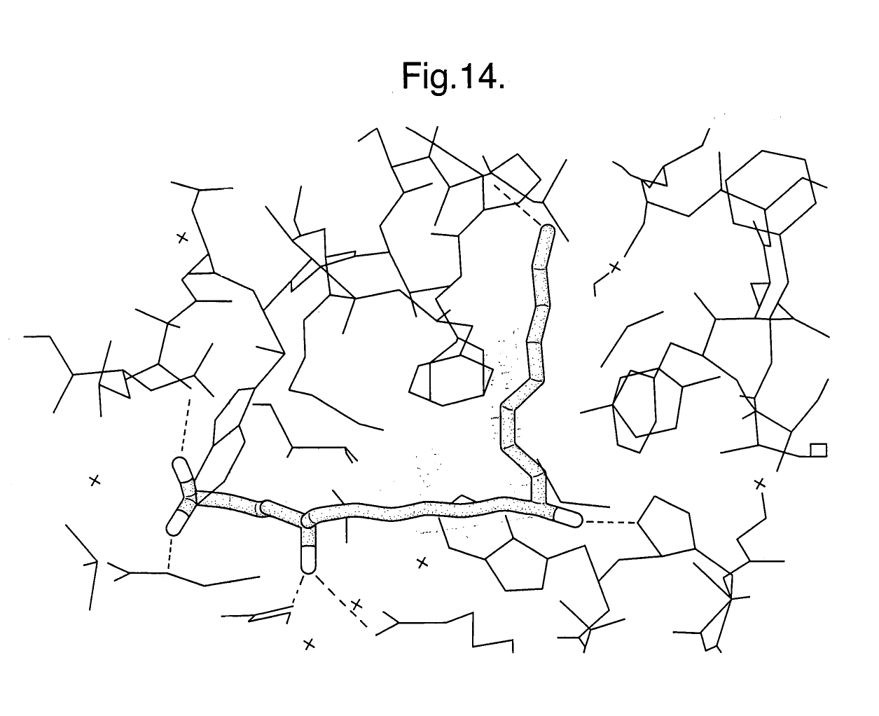

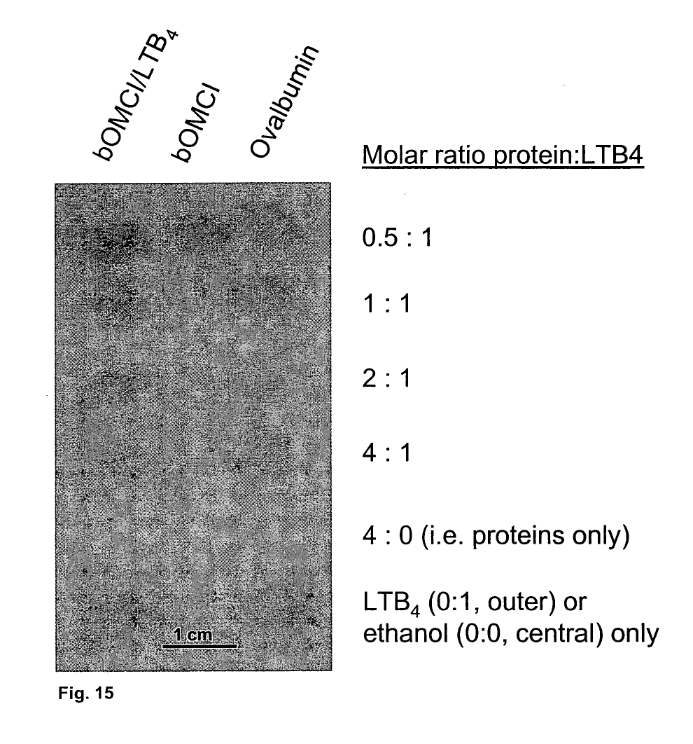

FIG. 14 shows a ball and stick representation of LTB4 in the bOMCI binding pocket. The following residues are directly involved in binding to LTB4:

Arg54,Thr85,Trp87: these residues hydrogen bond the head (carboxy group) of LTB₄; modifications of these residues can be engineered to bind ligands that differ in the chemistry of the head group

The hydrophobic body of the LTB4 contacts the hydrophobic side chains of the pocket: Phe36, Tyr43, Pro61, Leu70, Val72, Phe76, Leu57, Met74, Arg107, Phe89, Trp133, Trp87, Gly59

Arg107 and Gln105 recognise the —OH at LTB4 carbon 5 (C5)

His119 and Asp121 recognise the —OH at LTB4 carbon 12 (C12)

Ricinoleic acid lacks the —OH group at carbon 5, has only a single double bond between C9 and C10 and is two carbon atoms shorter than LTB4. The major structural differences between OMCI bound to ricinoleic acid bound compared to LTB4 are in the region of the 132-142 loop that is necessary for C5 inhibition (Mans and Ribeiro, 2008). The differences can be summarised as follows:

Glu141 and His164 side chains flip (these changes at His164 and Glu141 are related via two hydrogen bonds from the side chains of Arg47 and Arg148); as a result of these side chain flips, the His164:Asp136 salt bridge is lost and the 132-142 loop is pulled in via a side chain flip of His117, which hydrogen bonds to G139, and loss of the bridging water. This conformational change induced by LTB4 binding may have an effect of the binding kinetics of OmCI to C5 but we do not presently have any direct evidence for this.

The second region which shows a minor rearrangement is 155-159 No direct contact exists between the CS-inhibitory region 132-142 and the pocket. The 132-142 loop structure is the same in all four copies in the asymmetric unit despite this loop being in three different crystal packing environments across the 4 copies: so it is possible that the differences with relation to the ricinoleic acid structure be due to subtle propagation of structure from ligand to loop via an intermediate layer of small changes.

### Example 8

### Pre-Loading bOmCI with LTB4 Prevents the Tick Protein from Inhibiting the Local Skin Reaction Induced by Topical Application of LTB4

### Background

OmCI binds to a single molecule of LTB4 with high affinity (see example 6 and 7 above). Therefore saturating the binding site with LTB4 should prevent OmCI inhibiting the skin reaction induced by topical application of LTB4 (see example 5 above).

### Method:

bOmCI with and without LTB4 loaded into the binding pocket (example 6) was used. 100 ng LTB4 (vol. 2 μL of 50 ng/mL stock) was mixed with 28 μL PBS containing either bOmCI:LTB4, bOmCI or negative control protein ovalbumin. Solutions were incubated for 10 minutes at room temperature then applied to the flexor surface of the forearm and air dried. The most concentrated protein solutions used (including 2 μL pure ethanol), and LTB4 in PBS were also applied on their own as negative and positive controls respectively. The deposits were occluded under a chamber which was removed after 6 hours. Skin reactions were observed from 12-96 hours.

### Results:

As shown in FIG. 15, OmCI ablated the skin reaction induced by topical application of 100 ng LTB4 at a 4:1 to a 1:1 ratio. Whereas OmCI preloaded with LTB4 did not prevent the skin reaction even when used at a 4:1 molar ratio. Lower molar ratios of OmCI (0.5:1 and less) had no effect on lesion formation compared to LTB4 alone. Ovalbumin had no inhibitory effect on lesion formation. None of the proteins applied without LTB4 induced skin reactions. The results indicate that bOmCI saturated with LTB4 is unable to bind additional LTB4 in solution.

### Example 9

### OmCI Inhibits Immune Lung Disease

### Method:

Recombinant OmCI was given at 0, 50, 100 and 250 μg intravenously together with 300 μg Ova containing 0.3% Evans blue (EB). The OmCI was used as expressed or preloaded with LTB4 (see example 6). MK886 is a leukotriene synthesis inhibitor. MK886 was administered with 300 μg Ova containing 0.3% Evans blue (EB) as a positive control.

Intranasal anti-Ova antibody application (150 μg/mouse) was administered 15 minutes after the administration of OmCI, OmCI preloaded with LTB4 or MK886.

### Results:

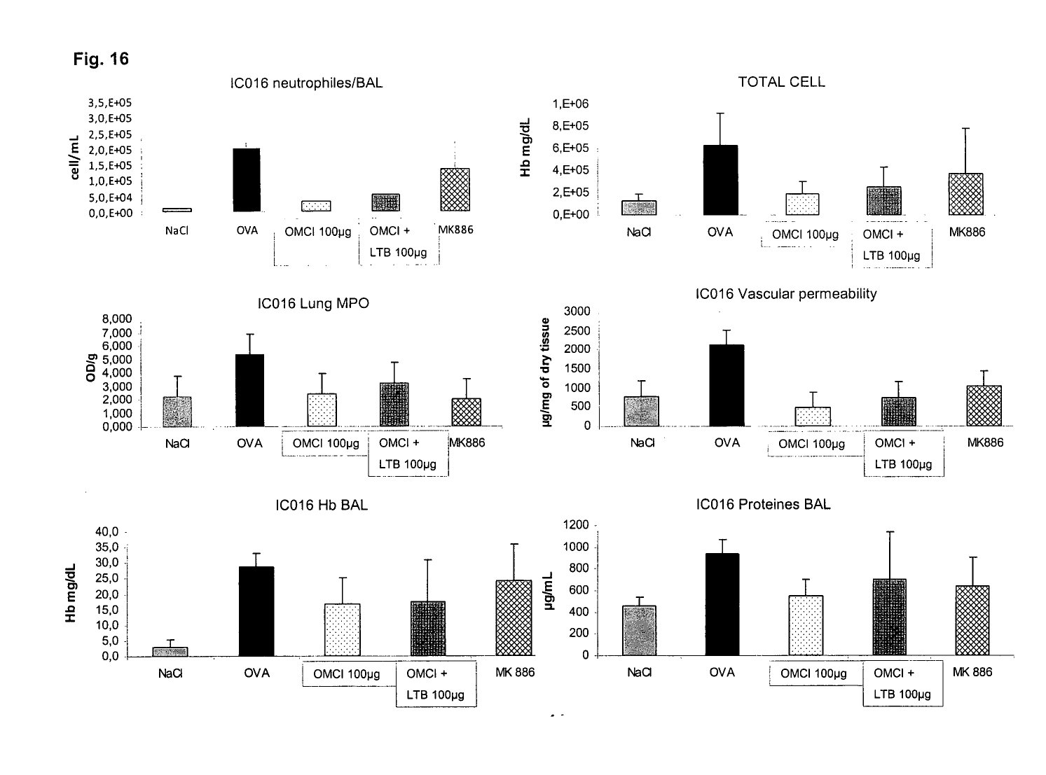

A dose of 100 μg OmCI injected intravenously at 15 min prior to the intranasal administration of OVA antibody reduced neutrophil recruitment in the lung and pulmonary microvascular damage with reduced protein exudations in the bronchoalveolar space (FIG. 16). The effect was dose dependent manner (data not shown).

Leukotriene B4 (LTB4) is produced locally upon immune complex induced lung injury, and inhibition of LTB4 with MK886 reduced dramatically the microvascular damage and inflammation (FIG. 16).

Structural data demonstrate that OmCI has an additional binding site for LTB4. Therefore we asked whether the LTB4 binding by OmCI might contribute to the inhibition of immune complex induced disease. Indeed, saturation of the LTB4 binding site attenuated the inhibitory effect of OmCI although it did not abrogate the response (FIG. 16).

### Discussion:

OmCI expresses functional C5 and LTB4 binding sites, and scavenging of immune complex induced C5 and LTB4 contributes to the inhibition of lung pathology.

### REFERENCES

Aiello, R. J., Bourassa P-A., Lindsey, S., Weng, W., Freeman, A., Showell, H. J. (2002). Leukotriene B4 Receptor Antagonism Reduces Monocytic Foam Cells in Mice. Arteriosclerosis, Thrombosis, and Vascular Biology 22, 443-449.

Aoki, H., Hisada, T., Ishizuka, T., Utsugi, M., Kawata, T., Shimizu, Y., Okajima, F., Dobashi, K., Mori, M. (2008) Resolvin E1 dampens airway inflammation and hyperresponsiveness in a murine model of asthma. Biochem Biophys Res Commun. 367:509-15.

Arita, M., Yoshida, M., Hong, S., Tjonahen, E., Glickman, J. N., Petasis, N. A., Blumberg, R. S., Serhan, C. N. (2005) Resolvin E1, an endogenous lipid mediator derived from omega-3 eicosapentaenoic acid, protects against 2,4,6-trinitrobenzene sulfonic acid-induced colitis. Proc Natl Acad Sci USA. 102:7671-6.

Aya, I. (2006) Blockade of leukotriene B4 signalling pathway directly inhibits cell proliferation and induces apoptosis colon cancer, Yokohama Medical Journal 57, 43-52.

Bisgaard H., Groth S., and Madsen F. (1985). Bronchial hyperreactivity to leukotriene D4 and histamine in exogenous asthma. Br Med J. 290, 1468-1471.

Bannenberg, G. L., Chiang, N., Ariel, A., Arita, M., Tjonahen, E., Gotlinger, K. H., Hong S. and Serhan, C. N. (2005). Molecular Circuits of Resolution: Formation and Actions of Resolvins and Protectins. J. Immunol. 174, 4345-4355.

Chen, M., Lam, B. K., Kanaoka, Y., Nigrovic, P. A., Audoly, L. P., Austen, K. F. and Lee D. M. (2006). Neutrophil derived leukotriene B4 is required for inflammatory arthritis. J. Exp. Med. 203, 837-842.

Chiang, N., Serhan, C. N., Dahlen, S-E., Drazen, J. M., Hay, D. W. P., Rovati, G. E., Shimizu, T., Yokomizo, T. and Brink, C. (2006). Review Article: The lipoxin receptor ALX: potent ligand-specific and stereoselective actions in vivo. Pharmacol Rev 58:463-487.

Cui, T., Schopfer, F. J., Zhang, J., Chen, K., Ichikawa, T., Baker, P. R. S., Batthyany, C., Chacko, B. K., Feng, X., Patel, R. P., Agarwal, Freeman, B. A., and Chen, Y. E. (2006) Nitrated Fatty Acids: Endogenous Anti-inflammatory Signalling Mediators. J. Biol. Chem. 281, 35686-35698.

Curry, S. L., Cogar, S. M. and Cook, J. L. (2005). Nonsteroidal Antiinflammatory Drugs: A Review. Journal of the American Animal Hospital Association 41, 298-309.

Curtis-Prior, P. (ed.) The Eicosanoids. John Wiley & Sons. ISBN 0471 1489840.

Czarnetzki, B. (1983). Increased monocyte chemotaxis towards leukotriene B4 and platelet activating factor in patients with inflammatory dermatoses. Clin Exp Immunol. 54, 486-492.

Del Prete A., Shao W. H., Mitola S., Santoro G., Sozzani S., and Haribabu, B. (2007) Regulation of dendritic cell migration and adaptive immune response by leukotriene B4 receptors: a role for LTB4 in up-regulation of CCR7 expression and function. Blood, 109, 626-631.

Ding, X-Z., Hennig, R. and Adrian T. E. (2003). Lipoxygenase and cyclooxygenase metabolism: new insights in treatment and chemoprevention of pancreatic cancer. Mol. Cancer 2, 10.

Drazen J. M. (1988). Comparative contractile responses to sulfidopeptide leukotrienes in normal and asthmatic human subjects. Ann NY Acad Sci. 524, 289-297.

Dubé L. M., Swanson L. J., Awni W. M., Bell R. L., Carter G. W., Ochs R. F. (1998). Zileuton: the first leukotriene inhibitor for use in the management of chronic asthma. In: Drazen J M, Dahlén S, Lee T H, eds. Five-lipoxygenase Products in Asthma. New York, N.Y.: Marcel Dekkar, Inc.

Ford-Hutchinson, A. (1990). Leukotriene B4 in inflammation. Crit. Rev. Immunol. 10, 1-12.

Freedman R. S., Wang E., Voiculescu S., Patenia R., Bassett R. L. Jr, Deavers, M., Marincola F. M., Yang P., Newman R. A. (2007). Comparative analysis of peritoneum and tumor eicosanoids and pathways in advanced ovarian cancer. Clin. Cancer Res. 13, 5736-5744.

Furstenberger, G., Krieg, P., Muller-Decker, K. and Habenicht A. J. (2005). What are cyclooxygenases and lipoxygenases doing in the driver's seat of carcinogenesis? Int. J. Cancer 119, 2247-2254.

Ghosh, J. and Myers, C. E. (1998). Inhibition of arachidonate 5-lipoxygenase triggers massive apoptosis in human prostate cancer cells. Proc Natl Acad Sci. USA 95, 13182-13187.

Gilroy, D. W., Lawrence, T., Perretti, M. and Rossi A. G. (2002). Inflammatory resolution: new opportunities for drug discovery. Nature Reviews 3, 401-416.

Greaves, M. W. (1984). Novel in vivo models of human skin pathophysiology. British Journal of Dermatology 111, Supplement 27, 183-187.

Griffiths, R., Pettipher, E., Koch, K., Farrell, C., Breslow, R., Conklyn, M., Smith,

M., Hackman, B., Wimberly, D., Milici, A., et al. (1995). Leukotriene B4 plays a critical role in the progression of collagen induced arthritis. Proc. Nat. Acad. Sci. U.S.A. 92, 517-521.

Hammamieh, R., Sumaida, D., Zhang, X. Y., Das, R. and Jett,. M. (2007). Control of the growth of human breast cancer cells in culture by manipulation of arachidonate metabolism. BMC Cancer 7: 138.

Hao C. M. and Breyer M. D. (2007). Physiologic and pathophysiologic roles of lipid mediators in the kidney. Kidney International 71, 1105-1115.

Harrison, K. A., Murphy, R. C. (1995). Isoleukotrienes are biologically active free radical products of lipid peroxidation. J. Biol. Chem. 270, 17273-17276.

Hasturk H., Kantarci A., Goguet-Surmenian E., Blackwood A., Andry, C., Serhan, C. N. and Van Dyke T. E. (2007). Resolvin E1 Regulates Inflammation at the Cellular and Tissue Level and Restores Tissue Homeostasis In Vivo. J. Immunol. 179, 7021-7029.

Hebell, T., Ahearn, J. M., and Fearon, D. T. (1991). Suppression of the immune response by a soluble complement receptor of B lymphocytes. Science, 254, 102-105.

Heller E. A., Liu E., Tager A. M., Sinha S., Roberts J. D., Koehn S. L., Libby P., Aikawa E. R., Chen J. Q., Huang P., Freeman M. W., Moore K. J., Luster A. D., and Gerszten R. E. (2005) Inhibition of atherogenesis in BLT1-deficient mice reveals a role for LTB4 and BLT1 in smooth muscle cell recruitment. Circulation 112, 578-586.

Hennig, R., Ventura, J., Segersvard, R., Ward, E., Ding, X-Z., Rao, S. M., Jovanovic, B. D., Iwamura, T., Talamonti, M. S., Bell, Jr, R. H. and Adrian, T. E. (2005). LY293111 Improves Efficacy of Gemcitabine Therapy on Pancreatic Cancer in a Fluorescent Orthotopic Model in Athymic Mice. Neoplasia 7, 417-425.

Hepburn, N. J., Williams, A. S., Nunn, M. A., Chamberlain-Banoub, J. C., Hamer, J., Morgan, B. P. and Harris, C. L. (2007) In vivo characterisation and therapeutic efficacy of C5-specific inhibitor from the soft tick Ornithodoros moubata. J Biol Chem. 282, 8292-8299.

Homeister, J. W., Satoh, P., and Lucchesi, B. R. (1992). Effects of complement activation in the isolated heart. Role of the terminal complement components. Circ. Res. 71, 303-319.

Hoover, H., Karnovasky, M., Austen, K., Corey, E. and Lewis, R. (1984). Leukotriene B4 action on endothelium mediates augmented neutrophil/endothelial adhesion. Proc. Nat. Acad. Sci. U.S.A. 81, 2191-2193.

Ikai, K. (1999). Psoriasis and the arachidonic acid cascade. J. Dermatol. Sci. 21, 135-146.

Imig, J. D. (2000). Eicosanoid regulation of the renal vasculature. Am. J. Physiol. Renal Physiol. 279, F965-F981.

Islam, S. A., Thomas, S. Y., Hess, C., Medoff, B. D., Means, T. K., Brander, C., Lilly, C. M., Tager, A. M., and Luster A. D. (2006). The leukotriene B4 lipid chemoattractant receptor BLT1 defines antigen-primed T cells in humans. Blood 107, 444-453.

Kallio, E., Lemstrom, K., Ryan, U., Hayry, P., and Koskinen P. (1999). Inhibition of complement reduces obliterative bronchiolitis. Transplantation Proceedings 31, 188.

Klaas P. J. M. van Gisbergen, Marta Sanchez-Hernandez, Teunis B. H. Geijtenbeek, and Yvette van Kooyk (2005) Neutrophils mediate immune modulation of dendritic cells through glycosylation-dependent interactions between Mac-1 and DC-SIGN. J. Exp. Med. 201, 1281-1292.

Kim, N. D., Chou, R. C., Seung, E., Tager, A. M. and Luster, A. D. (2006). A unique requirement for the leukotriene B4 receptorBLT1 for neutrophil recruitment in inflammatory arthritis. J. Exp. Med. 203, 829-835.

Liao T., Ke Y., Shao W. H., Haribabu B., Kaplan H. J., Sun D., and Shao H. (2006) Blockade of the interaction of leukotriene B4 with its receptor prevents development of autoimmune uveitis. Invest Opthalmol Vis Sci. 47, 1543-1549.

Lundeen K. A., Sun B., Karlsson L., and Fourie A. M. (2006) Leukotriene B4 receptors BLT1 and BLT2: expression and function in human and murine mast cells. J. Immunol. 177, 3439-3447.

Kim, N. D. and Luster, A. D. (2007). Regulation of immune cells by eicosanoid receptors. Review Article, The Scientific World Journal 7, 1307-1328.

McMahon, B. and Godson, C. (2004). Lipoxins: endogenous regulators of inflammation. Am J Physiol Renal Physiol. 286, F189-F201.

Mans, B. J. & Ribeiro, J. M. C. (2008) Function, mechanism and evolution of the moubatin-clade of soft tick lipocalins. Insect Biochem. Mol. Biol. 38:841-852.

Markiewski M. M., DeAngelis R. A., Benencia F., Ricklin-Lichtsteiner S. K., Koutoulaki A., Gerard C., Coukos G., Lambris J. D. (2008) Modulation of the antitumor immune response by complement. Nat Immunol. 9:1225-1235.

Massoumi, R., and Sjolander, A. (2007). The role of leukotriene receptor signaling in inflammation and cancer. The Scientific World Journal 7, 1413-1421.

Merched, A. J., Ko, K., Gotlinger, K. H., Serhan, C. N., Chan, L. (2008) Atherosclerosis: evidence for impairment of resolution of vascular inflammation governed by specific lipid mediators. FASEB J. 22:3595-606.

Miyahara, N., Miyahara, S., Takeda, K., and Gelfand G. W. (2006). Role of the LTB4/BLT1 Pathway in Allergen-induced Airway Hyperresponsiveness and Inflammation. Allergol Int. 55, 91-7.

Noiri, E., Yokomizo, T., Kakao, A., Izumi, T., Fujita, T., Kimura, S., and Shimizu, T. (2000). An in vivo approach showing the chemotactic activity of leukotriene B(4) in acute renal ischaemic-reperfusion injury. Proc Nat Acad Sci USA 97, 823-828.

Nunn, M. A., Sharma, A., Paesen, G. C., Adamson, S., Lissina, O., Willis, A. C., & Nuttall, P. A. (2005) Complement inhibitor of C5 activation from the soft tick Ornithodoros moubata J. Immunol. 174, 2084-2091.

Paesen G C, Adams P L, Harlos K, Nuttall P A, Stuart D I. (1999) Tick histamine-binding proteins: isolation, cloning, and three-dimensional structure. Mol Cell 3, 661-671.

Papayianni, A., Serhan, C. N. and Brady, H. R. (1996). Lipoxin A4 and B4 inhibit leukotriene-stimulated interactions of human neutrophils and endothelial cells. J. Immunol. 156, 2264-2272.

Peters-Golden, M. & Henderson Jr., W. R. (2007). Leukotrienes. N. Eng. J. Med. 357, 1841-1854.

Powell W. S., and Rokach J. (2005) Biochemistry, biology and chemistry of the 5-lipoxygenase product 5-oxo-ETE. Prog Lipid Res. 44, 154-183.

Rios-Santos, F., Benjamin, C. F., Zavery, D., Ferreira, S. H. and Cunha Fde, Q.

(2003). A critical role of leukotriene B4 in neutrophil migration to infectious focus in caecal ligation and puncture sepsis. Shock 19, 61-65.

Roversi, P. R., Lissina, O., Johnson, S., Ahmat, N., Paesen, G. C., Ploss, K., Boland, W., Nunn, M. A., and Lea, S. R. (2007) The structure of OmCI a novel lipocalin inhibitor of the complement system. J. Mol. Biol. 369:784-93.

Runarsson, G., Liu, A., Mahshid, Y., Feltenmark, S., Pettersson, A., Klein, E., Bjorkholm, M. and Claesson, H. (2005) Leukotriene B4 plays a pivotal role in CD40-dependent activation of chronic B lymphocytic leukemia cells. Blood 105: 1274-1279.

Samuelsson, B. (1983). Leukotrienes: mediators of immediate hypersensitivity reactions and inflammation. Science 220, 569-575.

Schwartz, G. K., Weitzman, A., O'Reilly, E., Brail, L., de Alwis, D. P., Cleverly, A., Barile-Thiem, B., Vinciguerra, V., and Budman D. R. (2005) Phase I and Pharmacokinetic Study of LY293111, an Orally Bioavailable LTB₄ Receptor Antagonist, in Patients With Advanced Solid Tumors. Journal of Clinical Oncology, 23, 5365-5373.

Sebaldt, R. J., Sheller, J. R., Oates, J. A., Roberts, L. J. and FitzGerald G. A. (1990).

Inhibition of eicosanoid biosynthesis by glucocorticoids in humans. Proc Natl Acad Sci. U.S.A. 8, 6974-6978.

Serhan, C. N. et al. (2000). Novel functional sets of lipid-derived mediators with antiinflammatory actions generated from omega-3 fatty acids via cyclooxygenase 2-nonsteroidal antiinflammatory drugs and transcellular processing. J. Exp. Med. 192, 1197-1204.

Serhan, C. N. et al. (2002). Resolvins: a family of bioactive products of omega-3 fatty acid transformation circuits initiated by aspirin treatment that counter proinflammation signals. J. Exp. Med. 196, 1025-1037.

Serhan C N and Savill J (2005) Resolution of inflammation: the beginning programs the end. Nat Immunol 6, 1191-1197.

Shao W. H., Del Prete A., Bock C. B., Haribabu B. (2006) Targeted Disruption of Leukotriene B4 Receptors BLT1 and BLT2: A Critical Role for BLT1 in Collagen-Induced Arthritis in Mice. J. Immunol. 176, 6254-6261.

Sharma, J. N. and Mohammed, L. A. (2006). The role of leukotrienes in the pathophysiology of inflammatory disorders: is there a case for revisiting leukotrienes as therapeutic targets? Immunopharmacology 14, 10-16.

Showell, H. J., Pettipher, E. R., Cheng, J. B., Breslow, R., Conklyn, M., Farrell, C. A, Hingorani, G. P., Salter, E. D., Hackman, B. C., Wimberly, D. J. et al (1995). The in vitro and in vivo pharmacologic activity of the potent and selective leukotriene B4 receptor antagonist CP-105696. J. Pharm. Exp. Ther. 273, 176-184.

Stelmach I., Korzeniewska A., Stelmach W., Majak P., Grzelewski T., Jerzynska J. (2005). Effects of montelukast treatment on clinical and inflammatory variables in patients with cystic fibrosis. Ann Allergy Asthma Immunol. 95, 372-380.

Tager, A. M. and Luster, A. D. (2003). BLT1 and BLT2: the leukotriene B(4) receptors. Prostaglandins Leukot. Essent. Fatty Acids 69, 123-134.

Takata, S., Matsubara, M., Allen, P. G., Janmey, P. A., Serhan, C. N. and Brady H. R. (1994). Remodeling of neutrophil phospholipids with 15(S)-hydroxyeicosatetraenoic acid inhibits leukotriene B4-induced neutrophil migration across endothelium. J Clin Invest. 93, 499-508.

Taube C., Miyahara N., Ott V., Swanson B., Takeda K., Loader J., Shultz L. D., Tager A. M., Luster A. D., Dakhama A., and Gelfand E. W. (2006) The leukotriene B4 receptor BLT1 is required for effector CD8+ T cell-mediated, mast cell-dependent airway hyperresponsiveness. J. Immunol. 176, 3157-3164.

Terawaki K., Yokomizo T., Nagase T., Toda A., Taniguchi M., Hashizume K., Yagi T., and Shimizu T. (2005) Absence of leukotriene B4 receptor 1 confers resistance to airway hyperresponsiveness and Th2-type immune responses. J. Immunol. 175, 4217-4225.

Ternowitz, Th., Andersen P. H., Bjerring P., Fogh K., Schroder J. M. and Kragballe K. (1989). 15-Hydroxyeicosatetraenoic acid (15-HETE) specifically inhibits the LTB4-induced skin response. Archives of Dermatological Res. 281, 401-405.

Van Dyke T. E. (2008) The management of inflammation in periodontal disease. (2008) J Periodontol. 79(8 Suppl): 1601-8.

Van Dyke, T. E. and Serhan, C. N. (2003). Concise review: Resolution of Inflammation: A New Paradigm for the Pathogenesis of Periodontal Diseases. J. Dent. Res. 82, 82-90.

Weringer, E. J., Perry, B. D., Sawyer, P. S., Gilman, S. C. and Showell, H. J. (1999). Antagonising leukotriene B4 receptors delays cardiac allograft rejection in mice. Transplantation 67, 808-815.

Yamaoka, K A., Claesson, H. E., and Rosen, A. (1989) Leukotriene B4 enhances activation, proliferation, and differentiation of human B lymphocytes. J. Immunol. 143, 1996-2000.

Yokomizo, T., Kato, K., Higiya, H., Izumi, T. and Shimizu, T. (1997). A G-protein coupled receptor for leukotriene B4 that mediates chemotaxis. Nature 387, 620-624.

Yokomizo, T., Kato, K., Terwaki, K., Y., Izumi, T. and Shimizu, T. (2000). A second leukotriene B(4) receptor, BLT2. A new therapeutic target in inflammation and immunological disorders. J. Exp. Med. 192, 421-432.

Yokomizo, T., Isumi, T., Chang, K., Takuwa, Y., Shimizu, T. (2001). Hydroxyeicosanoids bind to and activate the low affinity leukotriene B4 receptor BLT2. J. Biol. Chem. 276, 12454-12459.

Zhang, L., Zhang, X., Wu, P., Li, H., Jin, S., Zhou, X., Li, Y., Ye, D., Chen, B., Wan, J. (2008) BML-111, a lipoxin receptor agonist, modulates the immune response and reduces the severity of collagen-induced arthritis. Inflamm Res. 57:157-62.

Zhu, Y. I. and Stiller M. J. (2000). Preview of potential therapeutic applications of leukotriene B4 inhibitors in dermatology. Skin Pharmacol Appl Skin Physiol. 13, 235-245.

Ziboh, V. A., Miller, C. C., and Cho, Y. (2000). Metabolism of polyunsaturated fatty acids by skin epidermal enzymes: generation of antiinflammatory and antiproliferative metabolites. American J of Clin Nutrition 71, S361-S366.

1. An OmCI polypeptide or a polynucleotide encoding an OmCI polypeptide for the treatment of a disease or condition mediated by a leukotriene or hydroxyeicosanoid.

2. An OmCI polypeptide or polynucleotide according to claim 1 wherein said OmCI polypeptide is a tick derived complement inhibitor of Ornithodoros moubata, or a functional equivalent thereof which has leukotriene/hydroxyeicosanoid (LKJE) binding activity.

3. A polypeptide or polynucleotide according to claim 1, wherein said OmCI polypeptide comprises:
(a) an amino acid sequence of SEQ ID NO: 3;
(b) a variant thereof having at least 60% identity to the amino acid sequence of SEQ ID NO: 3 and LK/E binding activity; or
(c) a fragment of either (a) or (b) having LK/E binding activity.

4. A polypeptide according to claim 1 which binds to LTB4.

5. A polypeptide or polynucleotide according to claim 4 wherein said polypeptide consists of the sequence of SEQ ID NO: 2, or consists of the amino acids 19 to 168 of SEQ ID NO: 2.

6. A polynucleotide according to claim 1 wherein said polynucleotide comprises:
(a) the coding sequence of SEQ ID NO: 1;
(b) a sequence which is degenerate as a result of the genetic code to the sequence as defined in (a);
(c) a sequence having at least 60% identity to a sequence as defined in (a) or (b) and which encodes a polypeptide having LK/E binding activity; or
(d) a fragment of any one of the sequences as defined in (a), (b) or (c) which encodes a polypeptide having LK/E binding activity.

7. A polynucleotide according to claim 6 wherein said polynucleotide consists of the nucleic acid sequence shown in SEQ ID NO: 1.

8. A polypeptide or polynucleotide according to claim 1, wherein the disease or condition is selected from uveitis, atopic dermatitis, contact hypersensitivity, ulcerative colitis, oesophygeal adenocarcinoma, pancreatic adenocarcinoma, breast cancer, ovarian cancer, colon cancer, lung cancer, acne, obliterative bronchiolitis, aneurysm, periodontal disease, cystic fibrosis and prostate cancer, post-inflammatory pigmentation, fibromyalgia, systemic lupus erythematosus, tumor metastasis. sclerodermia, multiple sclerosis, sarcoidosis, radiation induced gastrointestinal inflammation, and gout.

9. A polypeptide or polynucleotide according to claim 1, wherein the disease or condition is selected from asthma, bronchitis, atherosclerosis, psoriasis, psoriatic arthritis, inflammatory bowel disease (including Crohn's disease), sepsis, arteritis, myocardial infarction, stroke, and coronary heart disease, ischaemia reperfusion injury, nephritis and arthritis, including rheumatoid arthritis, spondyloarthropathies, osteoarthritis, and juvenile arthritis,

10. A method of treating or preventing a disease or condition mediated by a leukotriene or hydroxyeicosanoid in a subject in need thereof, the method comprising administering to a subject a therapeutically effective amount of an OmCI polypeptide or a polynucleotide encoding an OmCI polypeptide.

11. A composition comprising an OmCI polypeptide and a fatty acid.

12. A method according to claim 10, wherein said OmCI polypeptide is selected from the group consisting of:
(a) an OmCI polypeptide that is a tick derived complement inhibitor of Ornithodoros moubata, or a functional equivalent thereof which has leukotriene/hydroxyeicosanoid (LK/E) binding activity;
(b) an OmCI polypeptide comprising:
(1) an amino acid sequence of SEQ ID NO: 3;
(2) a variant thereof having at least 60% identity to the amino acid sequence of SEQ ID NO: 3 and LK/E binding activity; or
(3) a fragment of either (1) or (2) having LK/E binding activity;
(c) an OmCI polypeptide that binds to LTB4;
(d) an OmCI polypeptide that binds to LTB4, and wherein the OmCI polypeptide consists of the sequence of SEQ ID NO: 2, or consists of the amino acids 19 to 168 of SEQ ID NO: 2.

13. A composition according to claim 11 for use in delivering the fatty acid to an individual.

14. A composition according to claim 11 wherein the fatty acid is selected from lipoxin A4, lipoxin B4, resolvins, protectins,15(S)-HETE, docosatrienes, 13-hydroxyoctadecadienoic acid, 15-hydroxyeicosatrienoic acid,15-hydroxyeicosapentaenoic acid, 17-hydroxydocosahexaenoic acid and nitrated fatty acids and analogues of any thereof.

15. A composition according to claim 11 for use in the treatment of inflammation.

16. A composition according to claim 15 for use in the treatment of uveitis, atopic dermatitis, contact hypersensitivity, ulcerative colitis, oesophygeal adenocarcinoma, pancreatic adenocarcinoma, breast cancer, ovarian cancer, colon cancer, lung cancer acne, obliterative bronchiolitis, aneurysm, periodontal disease, cystic fibrosis and prostate cancer, post-inflammatory pigmentation, fibromyalgia, systemic lupus erythematosus, tumor metastasis, selerodermia, multiple sclerosis, sarcoidosis, radiation induced gastrointestinal inflammation, and gout, asthma, bronchitis, atherosclerosis, psoriasis, psoriatic arthritis, inflammatory bowel disease (including Crohn's disease), sepsis, arteritis, myocardial infarction, stroke, and coronary heart disease, ischaemia reperfusion injury, nephritis and arthritis, including rheumatoid arthritis, spondyloarthropathies, osteoarthritis, and juvenile arthritis.
